(19) United States
(12) Patent Application Publication
(10) Pub. No.: US 2025/0191765 A1
(43) Pub. Date: Jun. 12, 2025

(54) METHOD FOR REAL-TIME RETREIVAL AND INTERPRETATION OF PATIENT DATA TO SUPPORT HEALTHCARE PROVIDERS DURING PATIENT ENCOUNTERS

(71) Applicant: Regard Technologies, Inc., Los Angeles, CA (US)

(72) Inventors: Daniel Lee Tryon, Los Angeles, CA (US); Kevin Hannigan, Los Angeles, CA (US)

(21) Appl. No.: 18/981,206

(22) Filed: Dec. 13, 2024

Related U.S. Application Data
(63) Continuation-in-part of application No. 18/756,869, filed on Jun. 27, 2024.
(60) Provisional application No. 63/609,752, filed on Dec. 13, 2023.
(60) Provisional application No. 63/636,019, filed on Apr. 18, 2024.
(60) Provisional application No. 63/524,508, filed on Jun. 30, 2023.
(60) Provisional application No. 63/524,495, filed on Jun. 30, 2023.

(51) Int. Cl. G16H 50/20; G16H 10/60

(52) U.S. Cl. CPC G16H 50/20; G16H 10/60

(57) ABSTRACT

One variation of a method for retrieving patient data for an encounter between a patient and a provider includes, at a first time during the encounter: receiving an indicator exhibited by the patient; querying a diagnostic model for a set of possible diagnoses for the patient based on the indicator; populating a first data request with a patient identifier, a set of evidence types supporting the possible diagnoses, and a time period; and rendering patient data—received responsive to the first data request—within the provider portal. This variation of the method further includes, at a second time succeeding the first time: populating a second data request with the patient identifier and the first time period; and rendering patient data—received responsive to the second data request—within the provider portal.

### CROSS-REFERENCE TO RELATED APPLICATIONS

This application claims the benefit of U.S. Provisional Application No. 63/609,752, filed on 13 Dec. 2023, which is incorporated in its entirety by this reference.

This application is a continuation-in-part of U.S. patent application Ser. No. 18/756,869, filed on 27 Jun. 2024, which claims the benefit of U.S. Provisional Application No. 63/636,019, filed on 18 Apr. 2024, U.S. Provisional Application No. 63/524,508, filed on 30 Jun. 2023, and U.S. Provisional Application No. 63/524,495, filed on 30 Jun. 2023, all of which are incorporated in their entireties by this reference.

### TECHNICAL FIELD

This invention relates generally to the field of health care and more specifically to a new and useful system and method for real-time identification and serving of patient data to health care providers in the field of health care.

### BRIEF DESCRIPTION OF THE FIGURES

FIGS. 1A, 1B, and 1C are flowchart representations of a method;

FIG. 2 is a flowchart representation of one variation of the method;

FIG. 3 is a flowchart representation of one variation of the method; and

FIGS. 4A and 4B are flowchart representations of one variation of the method.

### DESCRIPTION OF THE EMBODIMENTS

The following description of embodiments of the invention is not intended to limit the invention to these embodiments but rather to enable a person skilled in the art to make and use this invention. variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. METHOD

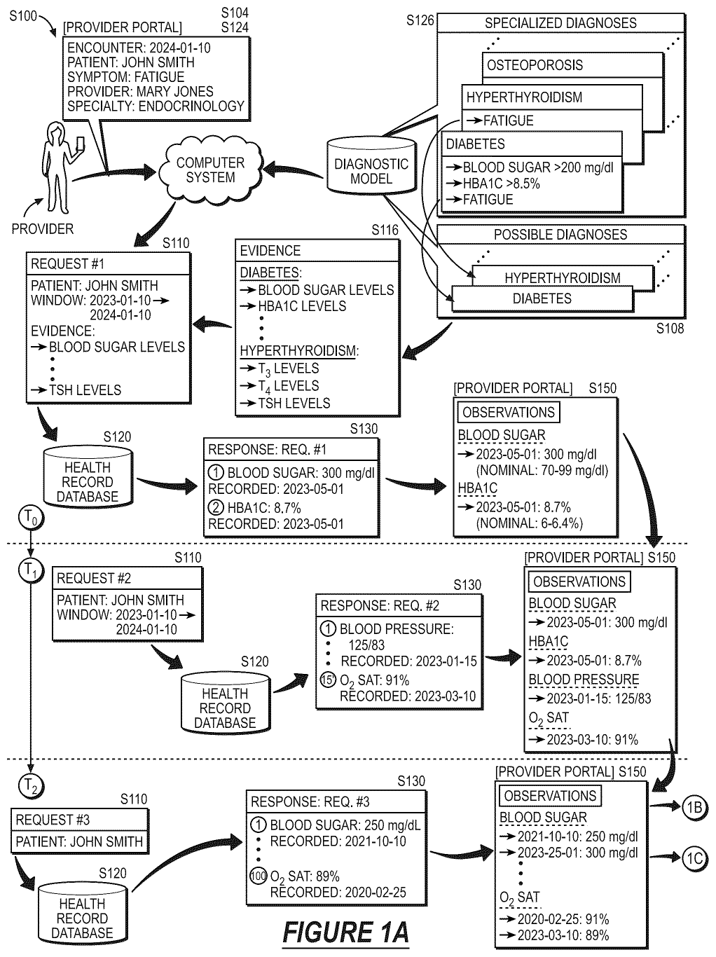

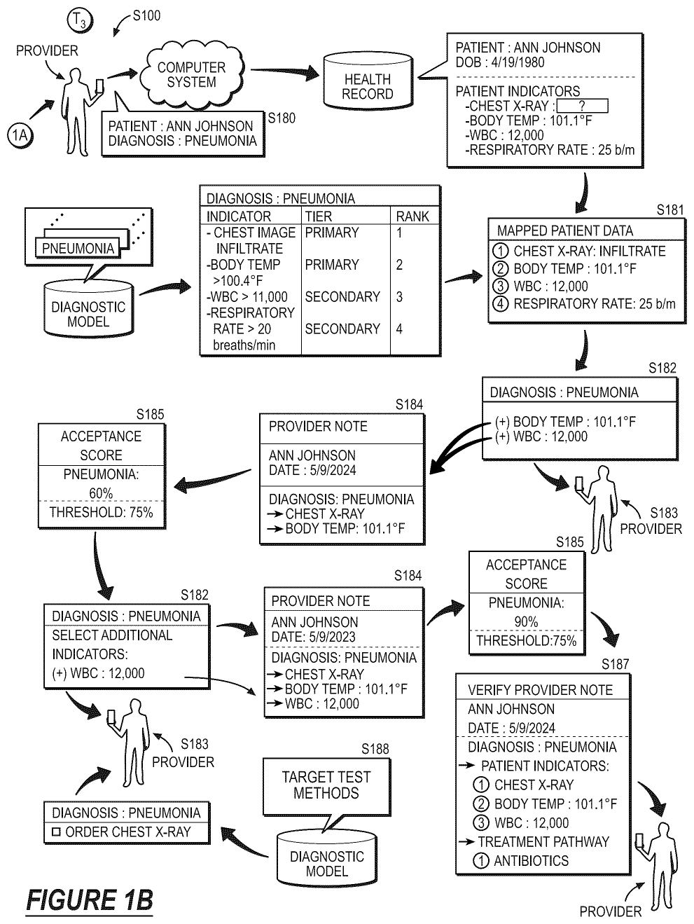

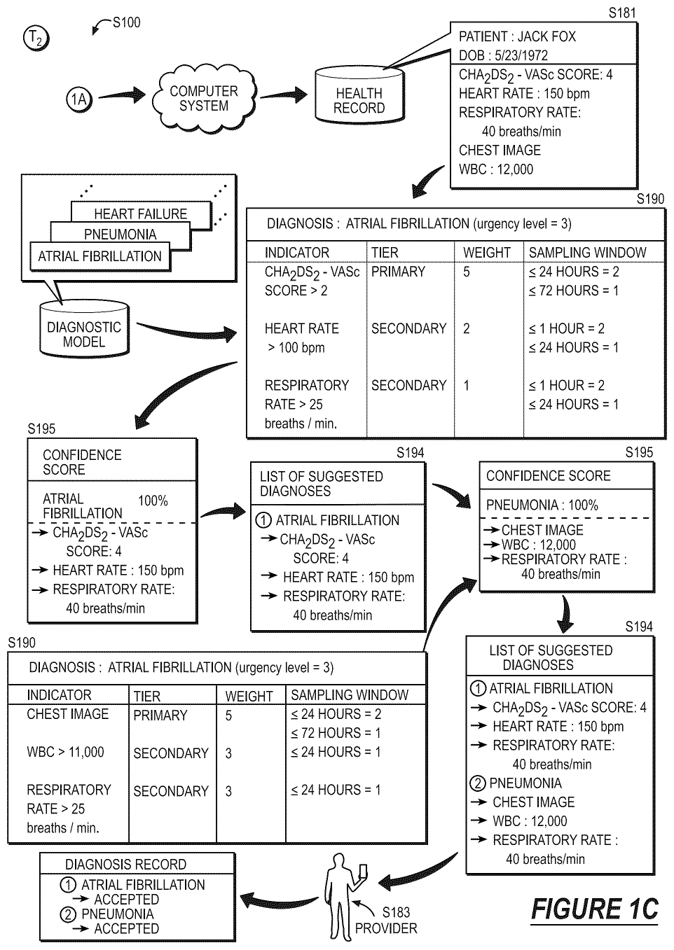

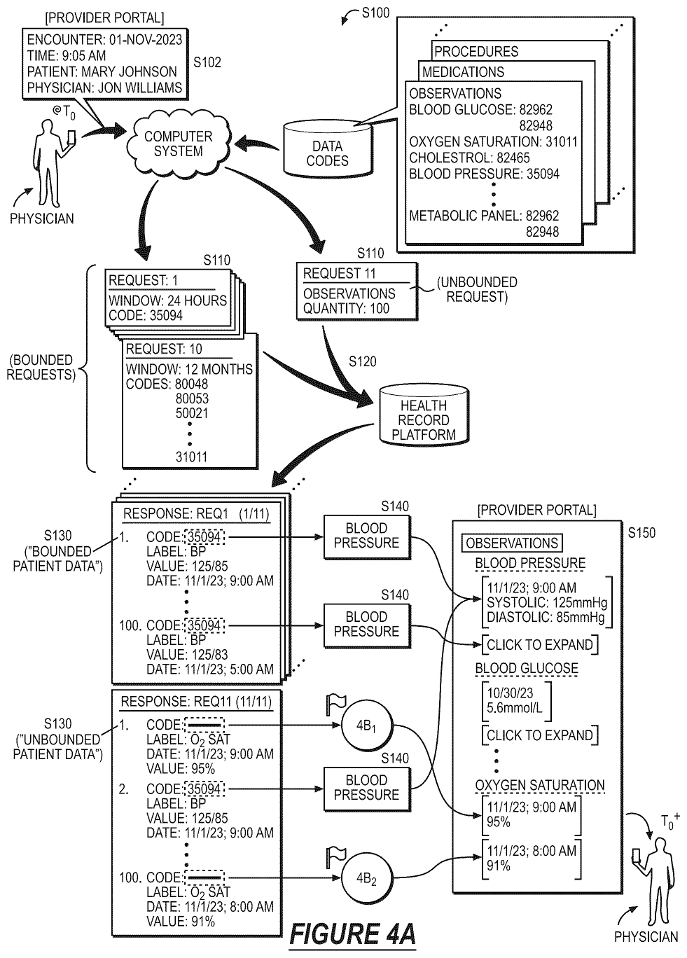

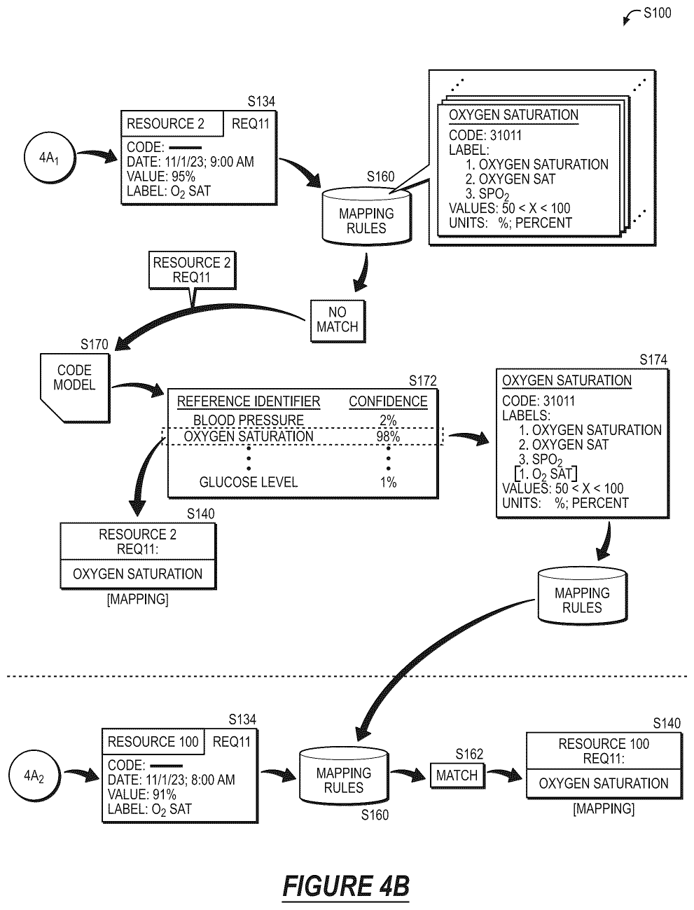

As shown in FIGS. 1A-1C, 4A, and 4B, a method S100 includes at a first time, during an encounter between a patient and a provider: receiving a first patient indicator exhibited by the patient during the encounter from the provider via a provider portal executing on a computing device accessed by the provider in Block S104; querying a diagnostic model for a first set of possible diagnoses in a population of diagnoses for the patient, each diagnosis in the first set of possible diagnoses associated with a set of evidence types analogous to the first patient indicator in Block S108; and aggregating a first set of evidence types corresponding to the first set of possible diagnoses in Block S116.

The method S100 further includes, at the first time: populating a first data request with a patient identifier of the patient, the first set of evidence types, and a first time period preceding the encounter in Block S110; transmitting the first data request to a health record database containing a corpus of historical patient data in Block S120; receiving a first set of patient data corresponding to the first set of evidence types responsive to the first data request in Block S130; and rendering the first set of patient data within the provider portal in Block S150.

The method S100 further includes, at a second time, succeeding the first time, during the encounter: populating a second data request with the patient identifier and the first time period in Block S110; transmitting the second data request to the health record database in Block S120; receiving a second set of patient data responsive to the second data request in Block S130; and rendering the second set of patient data within the provider portal in Block S150.

### 1.1 Variation: Pre-Encounter Data Request for Selective Patient Data

As shown in FIGS. 1A, 2, and 3, one variation of the method S100 includes, at a first time, preceding an encounter between a patient and a provider: accessing a patient identifier of the patient from a patient list representing patients associated with the provider via a provider portal executing on a computing device accessed by the provider in Block S102; populating a first data request with the patient identifier and a first time period preceding the encounter in Block S110; transmitting the first data request to a health record database containing a corpus of historical patient data in Block S120; and receiving a first set of patient data responsive to the first data request in Block S130.

This variation of the method S100 further includes, at the first time, rendering the first set of patient data within the provider portal by, for each unit of patient data in the first set of patient data: accessing a target format specified for the unit of patient data based on an evidence type corresponding to the unit of patient data; and populating a list of mapped patient data with the unit of patient data in the target format in Block S150.

This variation of the method S100 also includes, at a second time succeeding the first time, during the encounter with the patient: populating a second data request with the patient identifier, and a second time period defined between the first time and the second time in Block S110; transmitting the second data request to the health record database in Block S120; and receiving a second set of patient data responsive to the second data request in Block S130.

This variation of the method S100 further includes, at the second time, rendering the second set of patient data by, for each unit of patient data in the second set of patient data: accessing a target format specified for the unit of patient data based on an evidence type corresponding to the unit of patient data; and populating the list of mapped patient data with the unit of patient data in the target format in Block S150.

### 1.2 Variation: Pre-Encounter Data Request for All Patient Data

As shown in FIGS. 1A, 2, and 3, one variation of the method S100 includes, at a first time, preceding an encounter between a patient and a provider: accessing a patient identifier of the patient via a provider portal executing on a computing device accessed by the provider in Block S102; populating a first data request with the patient identifier in Block S110; transmitting the first data request to a health record database containing a corpus of historical patient data in Block S120; receiving a first set of patient data responsive to the first data request in Block S130; and rendering the first set of patient data within the provider portal by, for each unit of patient data in the first set of patient data, populating a list of mapped patient data with a unit of mapped patient data corresponding to the unit of patient data in Block S150.

This variation of the method S100 further includes, at a second time succeeding the first time, during the encounter with the patient: populating a second data request with the patient identifier and a time period defined between the first time and the second time in Block S110; transmitting the second data request to the health record database in Block S120; receiving a second set of patient data responsive to the second data request in Block S130; and rendering the second set of patient data by, for each unit of patient data in the second set of patient data, populating the list of mapped patient data with a unit of mapped patient data corresponding to the unit of patient data in Block S150. This variation of the method S100 further includes, at approximately the second time, serving the list of mapped patient data to the provider via the provider portal in Block S154.

### 1.2 Variation: Data Request Based on Evidence Types

As shown in FIGS. 4A and 4B, one variation of the method S100 includes, in response to receiving confirmation of initiation of an encounter between a patient and a provider via an instance of a provider portal accessed by the provider: receiving a set of encounter data—including a patient identifier associated with the patient—corresponding to the encounter in Block S102; populating a first data request for patient data with the patient identifier and a set of codes associated with a set of evidence types in Block S110; populating a second data request with the patient identifier and a target quantity of units of patient data in Block S110; at a first time, transmitting the first data request to a health record database containing a corpus of historical patient data in Block S120; and, at approximately the first time, transmitting the second data request to the health record database in Block S120.

This variation of the method S100 further includes, in response to transmitting the first data request to the health record database: receiving a first set of patient data corresponding to the set of codes defined in the first data request in Block S130; for each unit of patient data, in the first set of patient data, populating a list of mapped patient data with the unit of patient data linked to the evidence type in Block S150.

This variation of the method S100 further includes, in response to transmitting the second data request to the health record database, receiving a second set of patient data corresponding to the target quantity of units of patient data in Block S130. This variation of the method S100 further includes, in response to a first unit of patient data, in the second set of patient data, omitting a code in the set of codes: extracting a set of metadata from the first unit of patient data in Block S134; and accessing a set of mapping rules defined for patient data of a first evidence type in the set of evidence types in Block S160. In response to the set of metadata corresponding to criteria defined by the set of mapping rules, this variation of the method S100 further includes: assigning a first code—corresponding to the first evidence type—to the first unit of patient data in Block S140; and populating the list of mapped patient data with the first unit of mapped patient data in Block S150.

In response to the set of metadata falling outside criteria defined by the set of mapping rules, this variation of the method S100 further includes: accessing a rule-generating model configured to autonomously generate mapping rules based on metadata extracted from units of patient data in Block S170; based on the rule-generating model and the set of metadata, calculating a confidence score for the first evidence type paired with the first unit of patient data in Block S172; and, in response to the confidence score exceeding a threshold score, assigning the first evidence type to the first unit of patient data, generating a first mapping rule linking the set of metadata to the first evidence type, and appending the set of mapping rules with the first mapping rule in Block S174.

### 2. APPLICATIONS

Generally, the method S100 can be executed by a computer system (e.g., a remote computer system, a computer network, a remote server) in conjunction with a health record database (e.g., an electronic health record database) and/or application (e.g., native or web application): to receive a set of encounter data—including a patient identifier associated with a patient—at a start of an encounter between the patient and a health care provider (e.g., the patient's physician) via an instance of a provider portal (e.g., a native or web application) accessed by the health care provider (hereinafter the “provider”); to generate a data request for patient data collected for the patient; to selectively populate the data request with a set of predefined data codes, each predefined data code corresponding to a particular evidence type (e.g., vitals, lab results, conditions, medications, procedures, observations, provider notes, encounters) and/or with a set of evidence types; to transmit the data request to the health record database; and, within a target duration (e.g., less than five seconds) of the start of the encounter, receive a set of patient data—extracted from a health record database managed by the health record database—requested in the data request, such as corresponding to the set of predefined data codes and/or the set of evidence types.

The computer system can then: map units of patient data—contained in the set of patient data—to specific evidence types, in the set of evidence types, based on presence of predefined data codes in units of patient data and/or metadata (e.g., units, values, characters, template or structure, descriptions) extracted from these units of patient data; and assemble a list of mapped patient data—including accurately-labeled and relevant patient-specific data—accordingly. The computer system can then selectively surface this list of mapped patient data to the provider—and/or enable provider access to the list of mapped patient data—in (near) real-time, such that the provider may review this list at the start of the patient encounter. The computer system can therefore leverage this list of mapped data to quickly familiarize the provider with the patient's (relevant) medical history and/or key details regarding the current encounter with this patient.

### 2.1 Selective Data Requests

The computer system can selectively generate and transmit requests for patient data during different phases of a particular encounter to prioritize surfacing relevant patient data that supports immediate clinical decision-making for the provider. In particular, the computer system can selectively populate data requests with: different time periods (e.g., historical data from specific intervals such as one week, one month, or a year); and/or for different types of patient data (e.g., clinical data, medication history, test results). Additionally, the computer system can selectively transmit data requests at different times (e.g., before, upon initiation of, or during the encounter upon expiration of a delay period) throughout the encounter. The computer system can, therefore, dynamically adjust these data requests to minimize latency associated with retrieval of patient data while rendering a comprehensive health data record (e.g., including all patient data for the patient's lifetime) for the provider to review during the encounter.

In one example, upon initiation of an emergency encounter (e.g., an unscheduled encounter, an encounter within an emergency care facility) between a patient and a provider, the computer system: receives identification of a first patient; and receives selection of a symptom (or a “patient indicator”) exhibited by the patient from the provider, such as “chest pain.” The computer system then, at a first time coincident initiation of the emergency encounter: generates a first data request for all patient data recorded within the last 24 hours (e.g., to capture patient vitals recorded by an intake provider); and renders a first set of patient data—received in response to transmitting the first data request to the health record database—for the provider to review. The computer system then, at a second time succeeding the first time, during the emergency encounter: identifies a set of possible diagnoses for the patient and a set of evidence types supporting these possible diagnoses based on the chest pain symptom exhibited by the patient; generates a second data request for patient data—corresponding to the set of evidence types—recorded within the last 30 days (e.g., to capture patient data relevant to this symptom); and renders a second set of patient data—received in response to transmitting the second data request to the health record database—for the provider to review. Thus, in this example, the computer system strategically generates these data requests: to prioritize surfacing recently-recorded patient data that supports immediate clinical decision-making during the emergency encounter (e.g., patient vitals captured in the last 24 hours); and to subsequently surface historically-recorded patient data that adds context for the current symptom and aids in identifying trends or underlying conditions (e.g., recurring episodes of chest pain or related cardiac markers within the last 30 days).

In one example, prior to initiation of (e.g., ten minutes before) a follow-up encounter (e.g., a scheduled encounter) between a patient and a specialty provider (e.g., a cardiologist), the computer system: accesses a patient identifier of the patient (e.g., from the provider's appointment calendar); generates a first data request for patient data recorded within the last year and relevant to the provider's specialty (e.g., cardiac imaging results, blood pressure trends, and cholesterol panels); and renders a first set of patient data—received in response to transmitting the first data request to the health record database—for the provider to review in preparation for the encounter. The computer system then, at a second time coincident the start of the scheduled encounter: generates a second data request for all patient data recorded within the last 24 hours (e.g., to capture patient vitals recorded by an intake provider); and renders a second set of patient data—received in response to transmitting the second data request to the health record database—for the provider to review during the encounter. Thus, in this example, the computer system strategically generates these data requests: to generate a comprehensive view of historical patient data that is most relevant to the provider's specialty prior to the encounter (e.g., to enable informed review of long-term trends or previously flagged conditions); and to surface the most recent patient data that supports clinical decision-making during the encounter (e.g., patient vitals recorded on the day of the scheduled encounter).

### 2.2 Supporting Provider-Supplied Diagnoses+Predicting Diagnoses

Furthermore, the computer system can leverage a diagnostic model—linking a population of diagnoses to types of evidence supporting each diagnosis in the population of diagnoses—in combination with the list of mapped patient data to selectively surface units of patient data supporting a particular diagnosis supplied by the provider and/or to selectively surface possible diagnoses for a patient. In particular, in one implementation, the computer system can: receive identification of a diagnosis from the provider via the provider portal; retrieve a corresponding module, in the population of modules, defining the diagnosis specified by the provider; and identify a set of target evidence—defined by the module and corresponding to a set of target evidence types—supporting the diagnosis. The computer system can then: scan the list of mapped data for units of patient data corresponding to the set of evidence types; extract a set of units of patient data—corresponding to the set of evidence types—from the list; and selectively surface this set of units of patient data—paired with the diagnosis—to the provider within the provider portal. The computer system can thus interface with the provider portal to: assemble the list of mapped patient data at the start of the encounter; receive the diagnosis from the provider during the encounter; and, in (near) real-time, selectively present a list of evidence and/or combinations of evidence—extracted from the list of mapped patient data—that support the diagnosis to the provider.

### 3. PROVIDER PORTAL+PROVIDER NOTES

Generally, the computer system can host or interface with a provider portal (e.g., a native application or web application) executing on a computing device (e.g., a mobile device, a computer) accessed by a provider to selectively support the provider in treating and/or diagnosing a patient and/or generating provider notes for a patient encounter.

In one implementation, the computer system can integrate with a database affiliated with a health care system (e.g., a hospital or hospital system, a clinic, a health care group)—such as an electronic health record (or “EHR”) database—to access health records (e.g., EHRs) containing historical and/or current health information of patients affiliated with the health care system. For example, the computer system can integrate with an EHR database affiliated with the health care system and therefore access a population of health records corresponding to a population of patients affiliated with the health care system, each health record, in the population of health records, containing timeseries patient data captured for a patient, in the population of patients, over time and including: a list of timestamped encounters for this patient at a facility affiliated with the health care system; a series of timestamped notes—each note corresponding to a timestamped encounter in the list of timestamped encounters—previously generated for this patient; a list of timestamped medications prescribed and/or employed by the patient (e.g., historically and/or currently); a series of timestamped vital data (e.g., heart rate, respiratory rate, body temperature, oxygen saturation) recorded for the patient; a series of timestamped test results—including labs and/or images—obtained for the patient; a list of timestamped medical diagnoses or symptoms exhibited by the patient; etc.

### 4. DIAGNOSIS MODULES

Generally, the computer system can implement a diagnostic model linking diagnoses to supporting evidence that support these diagnoses. In particular, the computer system can implement a diagnostic model including a set of modules corresponding to a set of diagnoses, such that each module, in the set of modules, corresponds to a particular diagnosis in the set of diagnoses. Each module, in the set of modules, can define a particular diagnosis and a set of evidence (or “indicators”)—such as corresponding to various evidence types—associated with (e.g., supporting) the particular diagnosis. For example, the diagnostic model can include a module defining a particular diagnosis and a set of evidence—predicted to indicate a positive diagnosis for the particular diagnosis—including: a first evidence type (e.g., a qualitative and/or quantitative evidence type), such as a blood pressure reading, a body temperature, a white blood cell count, a culture test, a scan analysis, etc.; and a target value (e.g., a quantitative value, a qualitative value) and/or target range of values for the first evidence type.

In particular, in one example, the diagnostic module can include a first module defining a first diagnosis of pneumonia and a first set of evidence—such as including a body temperature within a target temperature range, a white blood cell count within a target count range, an oxygen saturation exceeding a threshold oxygen saturation, a chest image (e.g., a target chest x-ray, a target chest CT) depicting a first set of target features, a procalcitonin level exceeding a threshold level, etc.—associated with pneumonia. The population of modules can further include a second module defining a second diagnosis of acute, systolic, congestive heart failure and a second set of evidence—such as including a left ventricular ejection fraction (or “LVEF”) exceeding a threshold fraction, a body weight exceeding a threshold body weight, a B-type natriuretic peptide (or “BNP”) level exceeding a threshold peptide level, a chest x-ray depicting a second set of target features, etc.—associated with acute, systolic, congestive heart failure. Furthermore, in this example, the population of modules can include a third module defining a third diagnosis of Type II Diabetes Mellitus (or “adult-onset diabetes”) and a third set of evidence—such as including a glucose level exceeding a threshold glucose level, a hemoglobin A1C (or “HbA1c”) level exceeding a threshold hemoglobin A1C level, a diabetic neuropathy level (e.g., specified by the patient) corresponding to a target neuropathy level—associated with Type II Diabetes Mellitus.

The computer system can therefore leverage this diagnostic model—in combination with historical patient data (e.g., medical records, lab results, provider notes, conditions, procedures, medications, observations, reports, encounters) retrieved for a particular patient—to: selectively present patient data supporting a diagnosis supplied by a provider via the provider portal; and/or selectively suggest diagnoses to the provider for a particular patient. The computer system can, therefore, access patient data—such as during and/or at a start of an encounter between the patient and the provider—corresponding to evidence types defined in the diagnostic model and feed this patient data into corresponding modules of the diagnostic model accordingly.

### 5. PATIENT DATA QUERIES

Blocks of the method S100 recite: populating a data request with a patient identifier of the patient, a set of evidence types, and a time period preceding the encounter in Block S110; transmitting the data request to a health record database containing a corpus of historical patient data in Block S120; receiving a set of patient data corresponding to the set of evidence types responsive to the data request in Block S130; and rendering the set of patient data within the provider portal in Block S150. Generally, in Block S120, the computer system can query a database for patient data for a particular patient prior to and/or during an encounter between the patient and a provider. In particular, in Block S110, the computer system can populate the data request with data parameters, such as patient information (e.g., a patient name), provider information (e.g., a provider name), patient indicators (e.g., symptoms exhibited by the patient), time periods (e.g., patient data recorded within one year of the encounter), etc.

For example, prior to an encounter, the computer system can access and/or receive a list of patients treated by a particular provider. The computer system can then identify a patient from the list of patients, and automatically generate and transmit a first data request for patient data (e.g., patient data recorded within a one-year time period preceding the encounter) corresponding to the patient. Then, in response to initiation of an encounter between the patient and the provider, the computer system can automatically generate and transmit a second data request for patient data (e.g., patient data recorded within 24 hours of the encounter) corresponding to the patient. Furthermore, during the encounter, the computer system can automatically generate and transmit a third data request for patient data (e.g., all patient data recorded during the patient's lifetime) corresponding to the patient. Thus, the computer system can preemptively render recently-recorded patient data prior to an encounter to enable the provider to access this data immediately upon initiation of an encounter. Furthermore, the computer system can subsequently retrieve and render additional, historical patient data during the encounter to enable the provider to access a comprehensive health data record for the patient.

### 6. PATIENT DATA QUERY PRIOR TO PATIENT ENCOUNTER

Block S102 of the method S100 recites accessing a patient identifier of the patient from a patient list representing patients associated with the provider via a provider portal executing on a computing device accessed by the provider. In one implementation, the computer system can generate and transmit a data request for patient data corresponding to this particular patient prior to initiation of an encounter between the patient and the provider, as shown in FIG. 1A. In particular, in this implementation, preceding the encounter between the patient and the provider, the computer system accesses a patient identifier of the patient from a patient list representing patients associated with (e.g., treated by) the provider. The computer system can then leverage the patient identifier to query the secure database for patient data associated with the patient and/or relevant to this encounter.

In particular, preceding the encounter, the computer system can: generate and transmit a data request for patient data corresponding to this particular patient; and render the patient data—received responsive to the data request—within the provider portal. For example, the computer system can: generate a first data request—containing the patient identifier—for patient data corresponding to the patient; at a first time (i.e., prior to an encounter), transmit the first data request to the secure database (e.g., a health record database); and render a first set of patient data within a first time window (e.g., one minute)—following receipt of the first set of patient data responsive to the first data request—for review by the provider. The computer system can therefore render the first set of patient data prior to an encounter between the patient and the provider, such that the provider may access this patient data immediately upon initiation of the encounter.

### 7. PATIENT DATA QUERY RESPONSIVE TO INITIATION OF PATIENT ENCOUNTER

In one implementation, the computer system can query the database for patient data for the patient in response to initiation of an encounter with this patient by a particular provider. For example, the provider may access the provider portal and select a patient profile associated with the patient, thereby triggering initiation of an encounter between the patient and this provider. Then, in response to initiation of the encounter within the provider portal, the computer system can automatically generate and transmit a set of requests for patient data corresponding to the patient.

In particular, in this implementation, in response to initiation of an encounter between the patient and the provider, the computer system receives a set of encounter data—corresponding to the particular encounter, patient, and/or provider—such as including: a patient identifier associated with the patient; a provider identifier associated with the provider; a timestamp corresponding to a start time of the encounter; and/or a data passcode (e.g., a dynamic passcode) configured to enable (temporary) access to patient data—stored in the health record database (e.g., a secure database remote from the computer system)—throughout a duration of the encounter. The computer system can then leverage the set of encounter data to query the secure database for patient data associated with the patient and/or relevant to this encounter.

In one implementation, the computer system can generate and transmit a set of parallelized or “concurrent” data requests for patient data corresponding to this particular patient. For example, in the preceding example, the computer system can: generate a second data request—containing the set of encounter data pointing to the patient—for patient data corresponding to the patient; and, at a second time (i.e., during the encounter) succeeding the first time, transmit the first data request to the secure database (e.g., a health record database). Additionally, the computer system can: generate a third data request—containing the set of encounter data pointing to the patient—for additional patient data corresponding to the patient; at approximately the second time, transmit the third data request to the health record database. The computer system can then render a second set of patient data and a third set of patient data within a second time window (e.g., five seconds)—following receipt of the second and third sets of patient data responsive to the second and third data requests—for review by the provider.

Accordingly, the computer system can generate and transmit the second and third data requests to this health record database approximately concurrently upon initiation of the encounter. Therefore, the computer system can render this additional patient data for the provider to access during the encounter.

### 8. PATIENT DATA QUERY DURING PATIENT ENCOUNTER

In one implementation, the computer system can query the database for patient data for the patient during the encounter between the patient and the provider. For example, in response to initiation of the encounter (e.g., via the provider portal), the computer system can: initiate a timer for a delay period (e.g., a predefined time period between successive requests); and automatically generate and transmit a data request for patient data in response to expiration of the delay period. In particular, during the encounter, the computer system can: generate and transmit a data request for patient data corresponding to this particular patient; and render the patient data—received responsive to the data request—within the provider portal.

For example, in the preceding example, the computer system can: generate a fourth data request for patient data corresponding to the patient; at a third time (i.e., during the encounter), transmit the fourth data request to the secure database (e.g., a health record database); and render a fourth set of patient data within a fourth time window (e.g., 30 seconds)—following receipt of the fourth set of patient data responsive to the fourth data request—for review by the provider. The computer system can therefore render the fourth set of patient data during the encounter to enable the provider to access a comprehensive health data record for the patient, while minimizing latency and ensuring timely retrieval of the more-recent patient data from the first, second, and third data requests.

Accordingly, to prevent overloading the health record database with data requests, the computer system can selectively transmit data requests for patient data: at different times (e.g., before, upon initiation of, or during the encounter upon expiration of a delay period); for different time periods (e.g., historical data from specific intervals such as one week, one month, or a year); and/or for different types of patient data (e.g., clinical data, medication history, test results).

Furthermore, the computer system can dynamically adjust the time windows for patient data received in response to different data requests to accommodate the varying urgency of the data needed during different phases of the encounter. For example, when more immediate data is required (e.g., critical health indicators), the computer system can implement shorter time windows (e.g., five seconds). Conversely, for less time-sensitive data (e.g., historical medication records), the system can implement longer processing times (e.g., two minutes). Therefore, the computer system can minimize latency associated with retrieval of patient data, while rendering a comprehensive health data record (e.g., including all patient data for the patient's lifetime) for the provider to review during the encounter.

### 8.1 Data Requests Specifying Medical Codes

Generally, the computer system can generate a set of data requests—such as including one or more (concurrent) data requests—for patient data of a particular set of evidence types (e.g., conditions, procedures, lab results, medications, observations, provider notes, encounters). In particular, in one implementation, in response to receiving the set of encounter data, the computer system can: initialize a data request specifying the set of encounter data; populate the data request with a set of data codes corresponding to a set of evidence types, such that each data code, in the set of data codes, corresponds to a particular evidence type in the set of evidence types; and transmit the data request to the health record database. The computer system can then receive a set of patient data (or “resources”)—such as contained in one or more data packets transmitted from the health record database—corresponding to the particular patient and the set of data codes (hereinafter “codes”) specified in the data request.

For example, the computer system can generate a request—containing the set of encounter data pointing to the patient, provider, and/or particular encounter—specifying one or more codes corresponding to one or more evidence types. In particular, one example, the computer system can generate the request specifying: a first code (e.g., a numerical or qualitative code) corresponding to a first evidence type (e.g., metabolic panel); a second code corresponding to a second evidence type (e.g., urinalysis); a third code corresponding to a third evidence type (e.g., blood count); etc. The computer system can then transmit this request to the health record database and receive a corresponding data packet—containing resources (e.g., units of patient data) corresponding to the first, second, and third codes—from the health record database, such as within seconds (e.g., less than 2 seconds, less than 5 seconds, less than 30 seconds) of transmitting the request.

In one implementation, the computer system can generate a unique request for each data class in a set of data classes (or “categories”). In this implementation, the computer system can then populate each request—corresponding to a particular data class (e.g., conditions, procedures, medications, observations)—with a set of data codes corresponding to a set of evidence types associated with the particular data class. For example, the computer system can: generate a first request for medication data; and populate the first request with a first set of codes corresponding to a particular set of medication types (e.g., antibiotics, pain relievers, vitamins). In this example, the computer system can also: generate a second request for lab data; populate the second request with a second set of codes corresponding to a particular set of lab types (e.g., blood tests, urinalysis, lipid panel); generate a third request for procedure data; and populate the third request with a third set of data codes corresponding to a particular set of procedure types (e.g., diagnostic procedures, surgical procedures, therapeutic procedures). The computer system can similarly populate requests for patient data corresponding to additional primary data types.

Additionally or alternatively, in another implementation, the computer system can generate a set of requests for patient data of a particular set of evidence types and captured during a target window, such as within a preceding 24-hour time period, within a preceding 1-month time period, within a lifetime of the patient, within a last encounter recorded for the patient, etc. For example, the computer system can generate a request—containing the set of encounter data pointing to the patient, provider, and/or particular encounter—specifying: a particular data class (e.g., observations, medications, labs); a first code corresponding to a first evidence type (e.g., blood pressure data) of the particular data class; a first time window (e.g., a 24-hour time window preceding the encounter) assigned to the first code; a second code corresponding to a second evidence type (e.g., body weight data) of the particular data class; a second time window (e.g., a five-year time window preceding the encounter) assigned to the second code; a third code corresponding to a third evidence type (e.g., smoking status); and a third time window (e.g., a lifetime of the patient) assigned to the third code.

The computer system can then transmit this request to the health record database and receive a corresponding data packet containing: a first set of patient data of the first evidence type captured during the first time window (e.g., a 24-hour time window preceding the encounter); a second set of patient data of the second evidence type captured during the second time window (e.g., a five-year time window preceding the encounter); and a third set of patient data of the third evidence type captured during the third time window (e.g., a lifetime of the patient). In particular, in this implementation, the computer system can selectively assign time windows to different evidence types to prioritize requesting high-relevancy and/or high-priority data over high-frequency data.

### 8.1.1 Populating Data Requests: Response Bin Size

In one implementation, the computer system can strategically populate a data request—generated for a particular encounter defining a particular patient and a particular provider—with a set of data codes (e.g., one data code, five data codes, twenty data codes, one-hundred data codes) to minimize latency between sending of the data request and receiving of patient data corresponding to this data request. In particular, the computer system can populate the data request with a set of codes—corresponding to a set of evidence types (e.g., a particular type of lab result, a particular procedure, a particular condition)—distributed across the data request in a particular distribution configured to minimize a quantity of data packets received in response to the data request, while maximizing a quantity of patient data contained in a single data packet.

In one example, the computer system receives data packets—containing patient-specific data—defining a threshold bin size (e.g., 100 units of patient data, 1 page of data). In this example, the computer system can strategically populate a first data request with a first set of codes corresponding to a first set of evidence types and predicted to return a first set of patient data defining a first bin size approximating—but not exceeding—the threshold bin size. Therefore, in response to transmitting the first data request, the computer system can receive the first set of patient data (e.g., from the health record database) in a first data packet, without requiring transmission of additional data packets in response to the first request. In this example, the computer system can also populate a second data request with a second set of codes corresponding to a second set of evidence types and predicted to return a second set of patient data defining a second bin size approximating—but not exceeding—the threshold bin size. In response to transmitting the second data request, the computer system can therefore receive the second set of patient data in a second data packet approximately concurrent receipt of the first data packet received in response to the first data request. In this example, the computer system can similarly populate and transmit a third, fourth, fifth, etc., (concurrent) data request.

The computer system can, therefore, populate this data request with codes configured to return data packets filled to and/or nearly filled to capacity (e.g., a threshold bin size), thereby minimizing a total quantity of data packets received and thus minimizing latency between sending of a data request and receiving of data packets in return. In one implementation, the computer system can selectively populate a data request with a set of codes—corresponding to a set of evidence types—based on frequency (e.g., predicted frequency) of occurrence of the set of evidence types within a corpus of patient data. In particular, in this implementation, the computer system can: access a data frequency defined for a particular evidence type; access a target time window defined for the particular evidence type; and predict a quantity of units of patient data—corresponding to the particular evidence type—within the target time window based on the data frequency. The computer system can then: repeat this process for each evidence type, in the set of evidence types, defined in the diagnostic model; and strategically populate a data request—with a set of codes corresponding to the set of evidence types—based on quantities of units of patient data predicted for the set of evidence types.

For example, the computer system can: access a first data frequency defined for a blood-pressure evidence type (e.g., a blood pressure reading); access a first target time window of 24 hours defined for the blood pressure evidence type; and predict a first quantity of units of blood-pressure data within a preceding 24-hour time window based on the first data frequency. The computer system can also: access a second data frequency defined for a medication type (e.g., medications administered, prescribed, and/or taken by the patient); access a second target time window of five years defined for the medication type; and predict a second quantity of units of medication data within a preceding five-year time window based on the second data frequency. Additionally, in this example, the computer system can: access a third data frequency defined for a lab type (e.g., lab results); access a third target time window of 30 days defined for the lab type; and predict a third quantity of units of lab data within a preceding 30-day time window based on the third data frequency.

The computer system can then access a threshold bin size defined for data packets—containing patient data—received from the health record database. Then, in response to the first quantity of units of blood-pressure data falling within a threshold deviation of the threshold bin size, the computer system can populate a first data request with a first code corresponding to the blood-pressure evidence type. In response to a summation of the second quantity of units of the medication type and the third quantity of units of the lab type falling within the threshold deviation of the threshold bin size, the computer system can populate a second data request with a second code—corresponding to the medication type—and a third code corresponding to the lab type. Alternatively, in response to the summation of the second quantity of units of the medication type and the third quantity of units of the lab type exceeding the threshold bin size—and, in response to the second quantity of units of the medication type falling outside the threshold deviation of the threshold bin size—the computer system can: populate the second data request with the second code corresponding to the medication type; and populate the second data request with a fourth code—corresponding to a different evidence type—in replacement of the third code. The computer system can then populate a third data request with the third code corresponding to the lab type and/or additional codes corresponding to additional evidence types.

Alternatively, in response to the summation of the second quantity of units of the medication type and the third quantity of units of the lab type falling outside the threshold deviation of the threshold bin size, the computer system can populate the second data request with: the second code corresponding to the medication type; the third code corresponding to the lab type; and a fourth code corresponding to a particular evidence type and defining a fourth quantity of units of the particular evidence type, such that a new summation of the second, third, and fourth quantities of data units falls within the threshold deviation of the threshold bin size.

### 8.1.2 Populating Data Requests: Request Length

Additionally or alternatively, in one implementation, the computer system can strategically populate a data request with a set of codes—corresponding to data of a set of evidence types—based on a length of the data request. In particular, in this implementation, the computer system can access a quantity of codes defined for each evidence type and strategically populate a data request with codes corresponding to a combination of evidence types based on these quantities.

In this implementation, the computer system can generate a data request including a pointer (e.g., a URL, an address, an identifier) to a particular set of patient data corresponding to a particular patient and contained in a secure database. The computer system can generate this data request such that the pointer includes a particular set of codes—corresponding to data of a set of evidence types—defined for the data request and therefore points to a subset of patient data, in the set of patient data corresponding to the particular patient, contained within the secure database. The computer system can therefore populate the data request with a particular combination of codes—corresponding to a combination of evidence types—configured to yield a pointer of a length less than a maximum pointer length.

In one example, the computer system can generate a first data request including a first pointer containing a set of procedure codes. In response to a length of the first pointer falling below the maximum pointer length, the computer system can append the first pointer with a set of medication codes in addition to the set of procedure codes. In another example, for an evidence type of hemoglobin data, the computer system can access a set of 1,000 hemoglobin codes corresponding to various types of hemoglobin data. In this example, the computer system can: generate a first data request including a first pointer—containing a first set of 500 hemoglobin codes—of a first length approximating and not exceeding the maximum pointer length; and generate a second data request including a second pointer—containing a second set of 500 hemoglobin codes different from the first set—of a second length approximating and not exceeding the maximum pointer length.

### 8.2 Data Requests Excluding Medical Codes

In one implementation, the computer system can additionally generate a data request for patient data—regardless of presence of codes corresponding to the evidence type—for a particular patient. For example, the computer system can: generate a first data request for patient data of a particular set of evidence types; generate a second data request—defining a threshold quantity of patient data (e.g., 100 units of patient data)—for the threshold quantity of units of patient data; and (approximately) concurrently transmit the first and second data requests to the health record database.

Then, in response to transmission of these data requests, the computer system can receive: a first set of patient data—each unit of patient data, in the first set of patient data, including a code corresponding to an evidence type of the unit of patient data—contained in a first set of data packets returned by the health record database; and a second set of patient data (e.g., corresponding to the threshold quantity and collected more recently than patient data omitted from the first set of patient data) contained in a second set of data packets returned by the health record database. In particular, the computer system can receive this second set of patient data regardless of whether patient data includes codes corresponding to the predefined set of evidence types.

The computer system can therefore transmit the second data request to access patient data omitted in the first set of patient data, such as due to omission of a code, in the set of predefined codes, and/or inclusion of a new code omitted from the set of predefined codes. For example, the computer system can receive the second set of patient including: a first subset of patient data omitting any codes; a second subset of patient data including a set of new codes omitted from the set of predefined codes; and a third subset of patient data including codes in the set of predefined codes, such that the third subset of patient data may include units of patient data also included in the first set of patient data.

### 8.2.1 Populating Data Requests: Time Period

In one implementation, the computer system can strategically populate a data request—generated for a particular encounter defining a particular patient and a particular provider—with a time period (e.g., a one-year time period preceding an encounter) to minimize latency between sending of the data request and receiving of patient data corresponding to the data request. More specifically, the computer system can strategically populate the data request with a time period to selectively surface patient data recorded for the patient within the time period.

In this implementation, the computer system can define a particular time period, such as a one-year time period to retrieve only the patient data (e.g., from the health record database) recorded within this time period. The computer system can then: populate a data request with the patient identifier and the time period; generate and transmit the data request to the health record database; and render the patient data—received responsive to the data request—within a particular time window for the provider to review (e.g., via the provider portal).

In one example, at a first time preceding an encounter between a patient and a provider, the computer system: accesses a patient identifier of the patient (e.g., from an upcoming appointment calendar associated with the provider); populates a data request with the patient identifier and an unbounded time period preceding the encounter; and generates and transmits the data request to the health record database. The computer system then, at approximately the first time, receives a set of patient data—in a corpus of patient data stored in a health record associated with the patient—for the patient (i.e., all patient data recorded for the patient prior to the encounter). Then, the computer system renders the set of patient data within a five-minute time window for the provider to review (e.g., via the provider portal). More specifically, the computer system can implement a particular time window (e.g., five minutes) to accommodate a larger data request (i.e., for all patient data). The computer system can therefore render a comprehensive set of patient data prior to an encounter to minimize delays for the provider upon initiation of the encounter.

In another example, at a first time coincident initiation of an encounter between a patient and a provider, the computer system: receives a patient identifier of the patient (e.g., from the provider); and populates a first data request with the patient identifier and a one-year time period preceding the encounter. The computer system then, at approximately the first time, receives a first subset of patient data for the patient responsive to the first data request. Then, the computer system renders the first subset of patient data—within a five-second time window following receipt of the first subset of patient data—for the provider to review (e.g., via the provider portal). Thus, at approximately the first time, the computer system rapidly retrieves and renders recent patient data (i.e., within the five-second time window) for the provider to review.

Additionally, in this example, at a second time succeeding the first time, during the encounter, the computer system populates a second data request with the patient identifier and a time period defined between the first time and the second time. The computer system then, at approximately the second time, receives a second subset of patient data for the patient responsive to the second data request. Then, the computer system renders the second subset of patient data—within a two-second time window following receipt of the second subset of patient data—for the provider to review (e.g., via the provider portal). Thus, at approximately the second time, during the encounter, the computer system rapidly retrieves and renders additional patient data (i.e., within the two-second time window), such as patient data recorded between a time of admittance to a healthcare facility and a time of evaluation by the provider.

Additionally, in this example, at a third time succeeding the second time, during the encounter, the computer system populates a third data request with the patient identifier and a time period preceding the one-year time period defined in the first data request. The computer system then, at approximately the third time, receives a third subset of patient data for the patient responsive to the third data request. Then, the computer system renders the third subset of patient data—within a two-minute time window following receipt of the third subset of patient data—for the provider to review (e.g., via the provider portal). Thus, at approximately the second time, during the encounter, the computer system retrieves and renders additional patient data omitted from the first and second data requests to enable the provider to review all patient data recorded for the patient (i.e., throughout the patient's lifetime).

Accordingly, the computer system can dynamically adjust time windows based on the urgency of the data request, thereby enabling rapid access to critical patient data during encounters. Therefore, the computer system can strategically populate data requests with time periods: to prioritize surfacing recent patient data, supporting immediate clinical decision-making; and to ensure access to comprehensive patient data while reducing latency associated with retrieving data during encounters.

### 8.2.2 Populating Data Requests: Evidence Type

Blocks of the method S100 recite: receiving a patient indicator exhibited by the patient during the encounter from the provider via a provider portal executing on a computing device accessed by the provider in Block S104; querying a diagnostic model for a set of possible diagnoses in a population of diagnoses for the patient, each diagnosis in the set of possible diagnoses associated with a set of evidence types analogous to the patient indicator in Block S108; aggregating a set of evidence types corresponding to the set of possible diagnoses in Block S116; and populating a data request with a patient identifier of the patient, the set of evidence types, and a time period preceding the encounter in Block S110.

In one implementation, in Block S110, the computer system can strategically populate a data request with a set of evidence types (e.g., lab test results, imaging studies, and clinical notes). More specifically, the computer system can strategically populate the data request with a set of evidence types—corresponding to data analogous to evidence supporting various diagnoses for the patient—based on patient indicators (e.g., symptoms, vital signs, or test abnormalities) exhibited by the patient

In this implementation, the computer system can receive a patient indicator exhibited by the patient during the encounter from the provider, such as a qualitative patient indicator (e.g., shortness of breath) and/or a quantitative patient indicator (e.g., elevated heart rate). The computer system can then: query a diagnostic model for a set of possible diagnoses in a population of diagnoses for the patient, each diagnosis in the set of possible diagnoses associated with a set of evidence types analogous to the patient indicator; and aggregate a set of evidence types corresponding to the set of possible diagnoses. More specifically, for each possible diagnosis, the computer system can identify a set of evidence types supporting the possible diagnosis (i.e., all evidence types supporting the possible diagnosis). The computer system can then aggregate these sets of evidence for each possible diagnosis into a composite set of evidence (i.e., the set of evidence). The computer system can then populate a data request with the patient identifier and the set of evidence types.

In one example, the computer system receives a patient indicator of a heart rate of 150 bpm exhibited by the patient during the encounter. The computer system then queries the diagnostic model for a set of possible diagnoses for the patient, each possible diagnosis associated with supporting evidence including a heart rate of 150 bpm. In particular, in this example, the diagnostic model returns the set of possible diagnoses including supraventricular tachycardia (or “SVT”) and sepsis.

The computer system then accesses a first set of evidence supporting an SVT diagnosis and including: an electrocardiogram (or “ECG”) reading indicating absence of P waves or narrow QRS complexes; an oxygen saturation level below 95%; a blood pressure reading depicting hypotension (e.g., systolic pressure <90 mmHg); and a heart rate exceeding 150 bpm. The computer system then accesses a second set of evidence supporting a sepsis diagnosis and including: a white blood cell count exceeding 12,000/μL or falling below 4,000/μL; a body temperature exceeding 101° F. or falling below 96.8° F.; a blood culture result indicating presence of bacterial pathogens; and a heart rate exceeding 150 bpm.

The computer system then: identifies a first set of evidence types corresponding to the first set of evidence supporting the SVT diagnosis and including electrocardiogram (or “ECG”) readings, oxygen saturation levels, blood pressure readings, and heart rate readings; identifies a second set of evidence types corresponding to the second set of evidence supporting a sepsis diagnosis and including white blood cell counts, body temperatures, blood culture results, and heart rate readings; aggregates the first and second sets of evidence into a set of evidence types (i.e., including all supporting evidence for both the SVT and sepsis diagnoses); and populates the data request with the patient identifier and the set of evidence types.

Accordingly, the computer system can: transform patient indicators into targeted data requests by linking the exhibited patient indicators to evidence types of possible diagnoses for the patient; and rapidly surface patient data relevant to the exhibited patient indicators for the provider to review (e.g., via the provider portal). Therefore, the computer system can strategically populate data requests with evidence types to prioritize surfacing relevant patient data that supports immediate clinical decision-making.

### 8.2.3 Populating Data Requests: Pre-Encounter Note

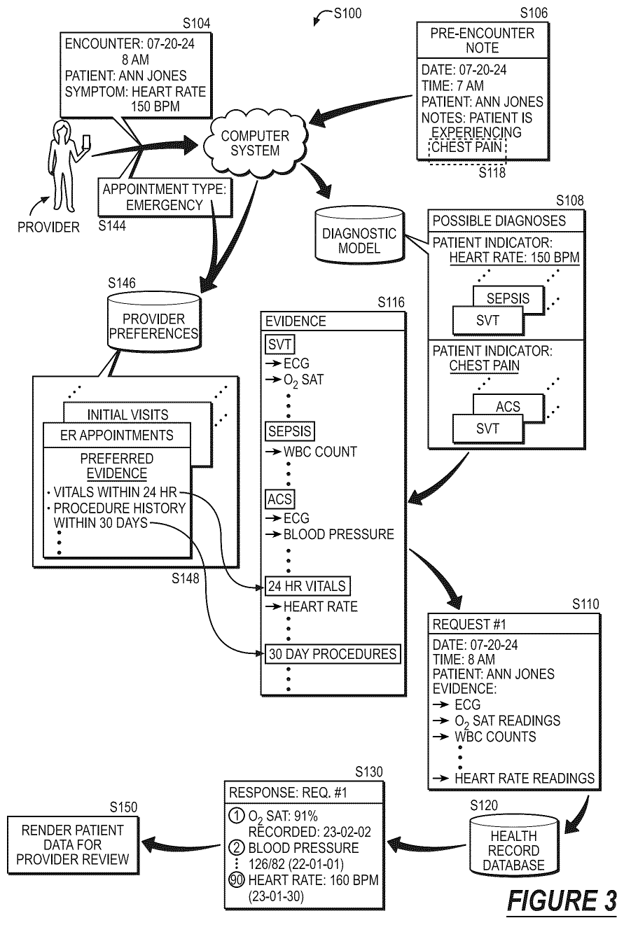

In one variation, as shown in FIG. 3, Blocks of the method S100 recite: accessing a pre-encounter note generated prior to encounter and associated with the patient in Block S106; and extracting a patient indicator from the pre-encounter note in Block S118. In one variation, the computer system can strategically populate a data request with a set of evidence types—based on patient indicator(s) exhibited by the patient and indicated in a pre-encounter note—to minimize latency between sending of the data request and receiving of patient data corresponding to the data request.

In this variation, the computer system can: access a pre-encounter note generated prior to the encounter (e.g., by the patient or an intake provider) and associated with the patient; and extract a first patient indicator from the pre-encounter note, such as a patient indicator (e.g., a symptom) described by the patient, or observed by an intake provider upon admittance of the patient to a healthcare facility. The computer system can then implement methods and techniques described above: to query the diagnostic model for a first set of possible diagnoses associated with a first set of evidence types analogous to the first patient indicator; to aggregate a first set of evidence types corresponding to the first set of possible diagnoses; and to populate a data request with the patient identifier and the first set of evidence types.

Additionally or alternatively, in response to receiving a second patient indicator exhibited by the patient (e.g., from the provider), the computer system can query the diagnostic model for a second set of possible diagnoses for the patient associated with a set of evidence types analogous to the second patient indicator. The computer system can then implement methods and techniques described above: to aggregate a second set of evidence types corresponding to the second set of possible diagnoses; and to populate the data request with the patient identifier and the second set of evidence types.

For example, in the preceding example, prior to the encounter, the computer system extracts a second patient indicator of “chest pain”—described by the patient—from a pre-encounter note. The computer system then queries the diagnostic model for a set of possible diagnoses for the patient, each possible diagnosis associated with supporting evidence, including chest pain. In particular, in this example, the diagnostic model returns the set of possible diagnoses, including SVT and acute coronary syndrome (or “ACS”).

The computer system then accesses a third set of evidence supporting the ACS diagnosis and including: a troponin level exceeding 0.04 ng/mL; an ECG reading indicating ST-segment elevation or depression; and a blood pressure reading depicting hypertension or hypotension. The computer system then: identifies a third set of evidence types corresponding to the third set of evidence supporting the ACS diagnosis and including troponin levels, ECG readings, and blood pressure readings; aggregates the first set of evidence (i.e., supporting the SVT diagnosis) and the third set of evidence into a second set of evidence types (i.e., including all supporting evidence for both the SVT and ACS diagnoses); and populates a second data request with the patient identifier, the second set of evidence types, and a one-year time period preceding the encounter. In response to receiving a set of patient data responsive to the second data request, the computer system renders the set of patient data (e.g., for the provider to review via the provider portal).

Then, during the encounter between the patient and the provider, the computer system implements methods and techniques described above: to receive the patient indicator of a heart rate of 150 bpm from the provider; to identify the second set of evidence types (i.e., including all supporting evidence sepsis diagnosis); and to populate the data request with the patient identifier, the second set of evidence types, and the one-year time period preceding the encounter. Thus, in this example, the computer system can rapidly retrieve and render patient data recorded within the previous year that supports possible diagnoses for the patient based on patient indicators (e.g., noted by the patient and/or recorded by the provider) exhibited by the patient.

### 8.2.4 Populating Data Requests: Provider Specialty

In one variation, Blocks of the method S100 recite: accessing a provider specialty associated with the provider in Block S124; and querying the diagnostic model for a set of specialized diagnoses, in the population of diagnoses, associated with evaluation and treatment by the provider based on the provider specialty in Block S126. In one variation, based on a patient indicator exhibited by the patient, the computer system can strategically populate a data request with a set of evidence types associated with a set of specialized diagnoses evaluated and treated by the provider.

In this variation, the computer system can: access a provider specialty associated with the provider (e.g., based on a provider identifier of the provider); and query the diagnostic model for a set of specialized diagnoses, in the population of diagnoses, associated with evaluation and treatment by the provider based on the provider specialty. The computer system can then: receive a patient indicator exhibited by the patient during the encounter from the provider; and, based on the patient indicator and the provider specialty, query the diagnostic model for a set of possible diagnoses intersecting the set of specialized diagnoses. In particular, the computer system can query the diagnostic model for the set of possible diagnoses, each diagnosis, in the first set of possible diagnoses: associated with a set of evidence types analogous to the patient indicator; and associated with evaluation and treatment by the provider.

In one example, the computer system: receives a qualitative patient indicator of “fatigue” exhibited by the patient during the encounter; and queries the diagnostic model for a set of possible diagnoses for the patient, each possible diagnosis associated with supporting evidence including fatigue. In particular, in this example, the diagnostic model returns the set of possible diagnoses including diabetes and hypertension. The computer system then: identifies a provider specialty of endocrinology associated with the provider; and queries the diagnostic model for a set of specialized diagnoses associated with evaluation and treatment by an endocrinologist (e.g., diabetes, osteoporosis, and hyperthyroidism). In particular, in this example, the diagnostic model returns a set of specialized diagnoses including diabetes and hyperthyroidism that intersect the set of possible diagnoses. The computer system then: accesses a first set of evidence supporting a diabetes diagnosis and including fatigue, a blood sugar reading exceeding 200 mg/dL, and an HbA1c level exceeding 8.5%; accesses a second set of evidence supporting a hyperthyroidism diagnosis and including fatigue, a thyroxine (or “T4”) level exceeding 1.8 ng/dL, a triiodothyronine (or “T3”) level exceeding 200 ng/dL, and a thyroid stimulating hormone (or TSH”) level falling below 0.1 mIU/L; identifies a first set of evidence types corresponding to the first set of evidence supporting the diabetes diagnosis and including blood sugar readings and HbA1c levels; identifies a second set of evidence types corresponding to the second set of evidence supporting the hyperthyroidism diagnosis and including thyroid hormone levels (T4 and T3) and TSH levels; aggregates a set of evidence types including the first and second sets of evidence types; and populates the data request with the patient identifier and the set of evidence types.

Accordingly, the computer system can: leverage provider specialties in addition to patient indicators to generate targeted data requests by linking the exhibited patient indicators to evidence types of specialized diagnoses treated by the provider; and rapidly surface patient data relevant to the exhibited patient indicators and the provider specialty (e.g., for the provider to review via the provider portal). Therefore, the computer system can strategically populate data requests with evidence types to prioritize surfacing relevant patient data that supports immediate clinical decision-making for the provider while minimizing irrelevant data and reducing cognitive load during the encounter.

### 8.2.5 Populating Data Requests: Encounter Type

In one variation, as shown in FIG. 3, Blocks of the method S100 recite: accessing an appointment type associated with the encounter in Block S144; and accessing a set of provider preferences specifying a set of evidence types historically queried by the provider for encounters of the encounter type in Block S148. In one variation, the computer system can strategically populate a data request with a set of evidence types based on an encounter type of a particular encounter between a patient and a provider, such as an initial encounter, a follow-up encounter, a pre-operative encounter, an emergency encounter, or an annual encounter.

In this variation, the computer system can access an encounter type associated with the encounter (e.g., from an upcoming appointment calendar associated with the provider) and/or receive the encounter type from a provider (e.g., upon initiation of the encounter). The computer system can then: access a provider profile, in a population of provider profiles, associated with the provider; and identify a set of provider preferences defining a set of evidence types based on the appointment type from the provider profile.

In one example, upon initiation of an initial visit between a patient and a provider (i.e., a first visit), the computer system accesses an initial encounter type from an upcoming appointment calendar associated with the provider. The computer system then: accesses a provider profile associated with the provider; and identifies a set of provider preferences defining a set of evidence prioritized by the provider for review during an initial encounter. In particular, in this example, the computer system identifies the set of evidence types including patient vitals, lab results, and procedures recorded for the patient within the last 30 days. The computer system then populates the data request with the patient identifier and the set of evidence types. Then, the computer system renders a set of patient data—received in response to transmitting the data request to the health record database—corresponding to the set of evidence types (e.g., for the provider to review).

Accordingly, the computer system can leverage provider preferences to generate targeted data requests that prioritize evidence types for review based on the encounter type. Therefore, the computer system can strategically populate data requests with evidence types to rapidly surface patient data relevant to a particular encounter type.

### 9. DATA MAPPING: TARGET FORMAT BASED ON EVIDENCE TYPE

Generally, the computer system can render patient data—received in response to transmitting a data request to the health record database—for the provider to review (e.g., via the provider portal). In particular, the computer system can render each unit of patient data in a target format based on an evidence type corresponding to the unit of patient data.

In one implementation, the computer system can implement methods and techniques described above to receive a set of patient data responsive to a data request. The computer system can then, for each unit of patient data in the set of patient data: access a target format specified for the unit of patient data based on an evidence type corresponding to the unit of patient data; and detect a format of the unit of patient data. The computer system can then populate a list of mapped patient data with units of patient data in response to detecting formats of these units of patient data congruent with target formats defined for these units of patient data. Alternatively, in response to detecting formats of units of patient data different from formats defined for these units of patient data, the computer system can: leverage a set of mapping rules—in combination with metadata extracted from units of patient data—to transform these units of patient data into the corresponding target formats; and then populate the list of mapped patient data with these units of patient data in the corresponding target formats.

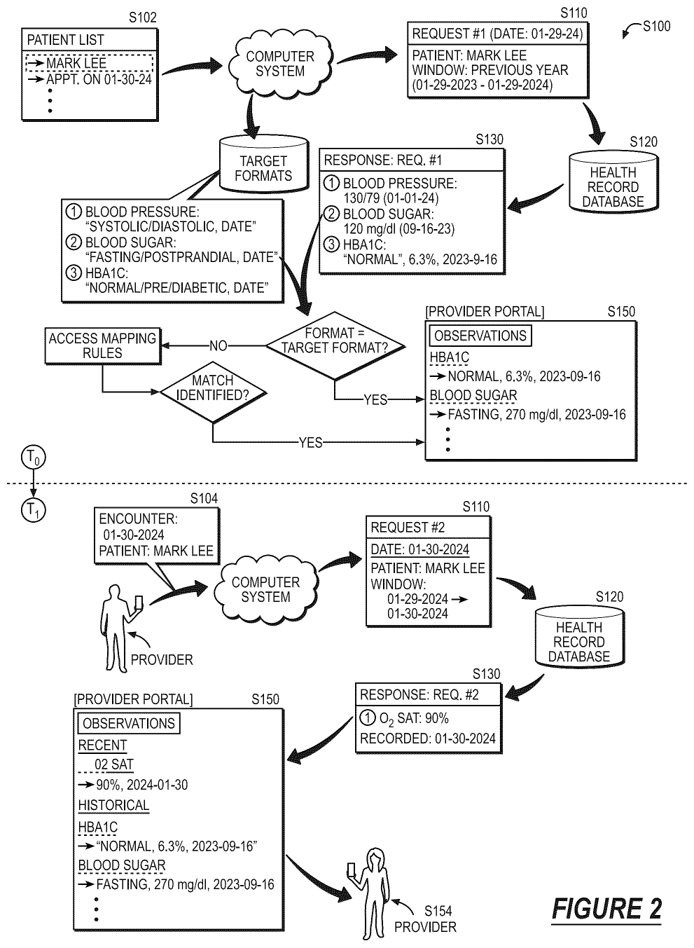

In particular, in this implementation, as shown in FIG. 2, for a first unit of patient data—in the set of patient data—corresponding to a first evidence type, the computer system can: detect a first format of the first unit of patient data congruent with a first target format specified for the first evidence type; and populate the list of mapped patient data with the first unit of patient data. For a second unit of patient data—in the set of patient data—corresponding to a second evidence type, the computer system can: detect a second format of the second unit of patient data different from a second target format specified for the second evidence type; extract a set of metadata from the second unit of patient data; and access a set of mapping rules defined for patient data of the second evidence type, in a corpus of evidence types, and stored in a rule database. Then, in response to the set of metadata corresponding to criteria defined by the set of mapping rules, the computer system can: transform the set of metadata into the second target format to generate a unit of mapped patient data; and populate the list of mapped patient data with the unit of mapped patient data.

In one example, for a unit of patient data corresponding to blood pressure data (i.e., the evidence type), the computer system accesses a target format specified for the blood pressure evidence type, such as presentation in the order “Blood Pressure Reading: Systolic/Diastolic, Date, Time” (e.g., “Blood Pressure Reading: 120/80 mmHg, 2024-11-19, 08:30 AM”). The computer system then: detects a format of the unit of patient data (e.g., “Systolic: 120, Diastolic: 80, Time: 08:30, Date: 2024 Nov. 19”) different from the target format; and extracts a set of metadata from the unit of patient data. The computer system then accesses a set of mapping rules defined for the blood pressure evidence type and including: a target evidence type (e.g., “Blood Pressure Reading”); a target unit (e.g., mmHg); a target timestamp format (e.g., “YYYY-MM-DD HH AM/PM”); and a target order (e.g., label first, followed by systolic/diastolic, and timestamp). Then, in response to the set of metadata corresponding to criteria defined by the set of mapping rules, the computer system can transform the set of metadata into the target format to generate a unit of mapped patient data.

Alternatively, in response to the set of metadata failing to correspond to criteria defined by the set of mapping rules, the computer system can: flag the unit of patient data for further investigation; and store the unit of patient data in a mapping queue for further investigation. For example, an administrator may access patient data in the mapping queue to manually review units of patient data and selectively transform these units of patient data. The administrator may then manually generate new mapping rules based on transformation of these units of patient data and metadata extracted from the units of patient data. Therefore, the computer system can transform patient data into target formats based on evidence types to render a comprehensive and standardized list of patient data for the provider to review.

### 10. DATA MAPPING: EVIDENCE TYPE BASED ON DATA CODE

In one variation, as shown in FIGS. 4A and 4B, Blocks of the method S100 include: in response to transmitting a data request to the health record database, receiving a set of patient data in Block S130; in response to a first unit of patient data, in the set of patient data, omitting a code in the set of codes, extracting a set of metadata from the unit of patient data in Block S134; accessing a set of mapping rules defined for patient data of a first evidence type in the set of evidence types in Block S160; and, in response to the set of metadata corresponding to criteria defined by the set of mapping rules, assigning a first code—corresponding to the first evidence type—to the first unit of patient data to the first unit of patient data to generate a first unit of mapped patient data in Block S140.

In one variation, the computer system can assign an evidence type to each unit of patient data to generate the list of mapped patient data. In particular, the computer system can assign a particular evidence type to a particular unit of patient data based on a code defined in the particular unit of patient data. The computer system can therefore: populate the list of mapped patient data with units of patient data assigned an evidence type and therefore including a code; and populate a list of unmapped patient data with units of patient data omitting a code and therefore not assigned to a particular evidence type.

In this variation, the computer system can leverage a set of mapping rules—in combination with metadata extracted from units of patient data—to selectively assign codes to unmapped patient data. In particular, in this variation, the computer system can: receive a first unit of patient data; and, in response to the first unit of patient data omitting a code, extract a first set of metadata from the first unit of patient data, such as including a title, a description, and/or a type of lab, procedure, medication, etc. The computer system can then access a first set of mapping rules defined for a first code, in the corpus of predefined codes, and stored in a rule database. For example, for an evidence type corresponding to blood pressure data, the computer system can leverage a set of mapping rules defined for the blood pressure evidence type and including: a particular code (e.g., 1234) corresponding to the blood pressure evidence type; a target text description (e.g., “blood pressure”); a set of expected units (e.g., mmHg); a set of expected characters or “data template” of “first whole number/second whole number”; an expected value or range of values (e.g., numerical and/or qualitative values) between 0 and 200; etc.

Based on the first set of metadata and the first set of mapping rules, the computer system can selectively assign and/or reject assignment of the first code to the first unit of patient data. In this variation, in response to assigning the first code to the first set of metadata, the computer system can: assign a first evidence type—corresponding to the first code—to the first unit of patient data; and append the list of mapped patient data with the first unit of patient data linked to the first evidence type. Alternatively, in response to rejecting assignment of the first code to the first unit of patient data, the computer system can: access a second set of mapping rules defined for a second code in the corpus of predefined codes; and, selectively assign and/or reject assignment of the second code to the first unit of patient data based on the first set of metadata and the second set of mapping rules accordingly. The system can repeat this process for each code, in the corpus of codes, to attempt to assign a particular code to the (unmapped) first unit of patient data.

Alternatively, in response to rejecting each code, in the corpus of codes, for assignment to the first unit of patient data, the computer system can: flag the first unit of patient data for further investigation; and store the first unit of patient data in a mapping queue for further investigation. For example, an administrator may access patient data in the mapping queue to manually review units of patient data and selectively assign codes to these units of patient data. The administrator may then manually generate new mapping rules based on assignment of codes to these units of patient data and metadata extracted from the units of patient data.

### 10.1 Real-Time, Automated Rule Generation

In one variation, Blocks of the method S100 include: in response to the set of metadata falling outside criteria defined by the set of mapping rules, accessing a rule-generating model configured to autonomously generate mapping rules based on metadata extracted from units of patient data in Block S170; based on the rule-generating model and the set of metadata, calculating a confidence score for the first evidence type paired with the first unit of patient data in Block S172; and, in response to the confidence score exceeding a threshold score, assigning the first evidence type to the first unit of patient data, generating a first mapping rule linking the set of metadata to the first evidence type, and appending the set of mapping rules with the first mapping rule in Block S174.

In one variation, the computer system can automatically predict an evidence type—corresponding to a code—of a particular data unit stored in the mapping queue based on a set of metadata extracted from the particular data unit. The computer system can then: automatically generate a new mapping rule linking characteristics of the set of metadata to the code; store this new mapping rule in the rule database; and implement the new mapping rule during mapping of subsequent patient data to automatically assign the code to units of patient data including metadata exhibiting equivalent and/or similar characteristics of the set of metadata.

In particular, in response to rejecting each code, in the corpus of codes, for assignment to the first unit of patient data, the computer system can: flag the first unit of patient data for further investigation; feed the first unit of patient data to a rule-generating model configured to automatically assign a particular code to the unit of patient data based on the first set of metadata; and generate a new mapping rule corresponding to the particular code and configured to link (future) units of patient data exhibiting metadata approximating and/or equivalent the first set of metadata—extracted from the first unit of patient data—to the particular code. The system can then append a set of mapping rules—defined for the particular code and stored in the rule database—to include the new mapping rule.

For example, the computer system can automatically generate new rules to associate an evidence type and/or code (e.g., corresponding to the evidence type) with a particular title, misspelled titles corresponding to the particular title, target values and/or ranges of values, possible units (e.g., “%”, “percent”), descriptors, etc. Later, the computer system can receive a second unit of patient data, and, in response to the second unit of patient data omitting a code: extract a second set of metadata from the second unit of patient data; and access the set of mapping rules stored in the rule database. Then, in response to the second set of metadata extracted from the second unit of patient data corresponding to a set of criteria defined by the new mapping rule—derived from the first unit of patient data—the computer system can automatically assign the particular code to the second unit of patient data. Then, in response to assigning the particular code to the second unit of patient data, the computer system can: selectively assign a particular evidence type—corresponding to the particular code—to the second unit of patient data; and append the list of mapped patient data with the second unit of patient data linked to the particular evidence type.

In one example, the computer system can: receive a unit of patient data—corresponding to hemoglobin data—omitting a data code and reciting “hg level 14 g/dl”; extract a set of metadata including a title of “hg level,” a value of “14,” and units of “g/dl”; and access the rule database to link the set of metadata to a particular evidence type. In this example, the rule database can define a first set of mapping rules—corresponding to the hemoglobin evidence type—defining: a set of titles including “hemoglobin,” “hemoglobin level,” and “Hgb,” and “Hgb level”; a value range of “5-20”; and units of “g/dl.” In response to the title of “hg level” differing from each title in the set of titles defined in the first set of mapping rules, the computer system can reject assignment of the hemoglobin evidence type to the unit of patient data. Then, in response to rejecting each other evidence type, in the set of evidence types, for assignment to the unit of patient data, the computer system can: flag the unit of patient data for further investigation; and access the rule-generating model to predict a particular evidence type for this unit of patient data. Based on a correlation between the set of metadata and the set of titles, value range, and units defined by the first set of mapping rules, the computer system can: predict a confidence score for the hemoglobin evidence type based on the rule-generating model and the set of metadata; assign the hemoglobin evidence type to the unit of patient data in response to the confidence score exceeding a threshold confidence score; and automatically update the first set of rules to include a new title of “hg” and “hg level” based on the set of metadata.

In one variation, the computer system: aggregates a population of data containers, each data container associated with a particular evidence type—corresponding to a particular code—paired with metadata (e.g., title, value, units, descriptors) extracted from patient data of the particular evidence type; and implements artificial intelligence, machine learning, regression, and/or other techniques to train a rule-generating model to predict evidence types of (unmapped) patient data defining various metadata based on the set of data containers. Then, in response to receiving a request for a predicted evidence type for a particular unit of patient data—including a set of metadata—the computer system can: initialize a data container (e.g., a vector, a matrix) for this unit of patient data; represent the set of metadata in slots of the data container; and feed this data container into the rule-generating model to output a predicted evidence type of the unit of patient data.

### 11. PRESENTATION OF MAPPED PATIENT DATA

In one variation, Blocks of the method S100 recite: receiving a set of patient data responsive to a data request transmitted to the health record database at a first time in Block S130; rendering the set of patient data within the provider portal in Block S150; and serving the list of mapped patient data to the provider via the provider portal at approximately the first time in Block S154.

The computer system can selectively serve the list of mapped patient data to the provider via the provider portal in (near) real time, such that the provider may quickly review the patient's relevant medical history at and/or within a threshold duration (e.g., 30 seconds) of a start of the patient encounter. For example, the computer system can sort the list of mapped patient data according to evidence type (e.g., via reference identifier), category (e.g., vitals, observations, medications, procedures), and/or relevancy to generate: a first list of patient data collected during a time period immediately preceding the encounter, such that the physician may quickly access and review new and/or most-recent patient data; a second list of patient data corresponding to vitals data captured for this patient; a third list of patient data corresponding to lab data received for this patient; a fourth list of patient data corresponding to medications currently and/or previously prescribed to this patient; a fifth list of patient data corresponding to historical procedures performed for this patient; etc. The computer system can then: generate a notification including a link—configured to trigger rendering of a window loaded with these lists of patient data—and indicating availability of patient data via selection of the link; and serve this notification to the provider via the provider portal. The provider may then select the link to selectively view each list of patient data within the window.

### 12. SUPPORTING PROVIDER-SUPPLIED DIAGNOSIS

In one variation, as shown in FIG. 1B, Blocks of the method S100 recite: receiving a diagnosis for the patient from the provider via the provider portal, the diagnosis associated with a set of target evidence types supporting the diagnosis in Block S180; extracting a set of patient data—from a list of mapped patient data—corresponding to the set of target evidence types in Block S181; generating a notification including the set of patient data and a prompt to review each unit of patient data, in the set of patient data, for insertion into a provider note generated for the encounter and indicating the diagnosis for the patient in Block S182; and transmitting the notification to the provider via the provider portal in Block S183.

In one variation, the computer system can insert the list of mapped patient data into modules of the diagnostic model to identify evidence—or units of patient data—that support diagnoses provided by the provider and/or to selectively suggest new diagnoses (e.g., not previously-provided by the provider). In particular, in response to receiving a first diagnosis for the patient from the provider via the provider portal, the computer system can: access a first module, in the population of modules, of the diagnostic model associated with the first diagnosis; extract a first set of target evidence types defined for the first diagnosis and corresponding to a first set of evidence types; access the list of mapped patient data generated for the patient; scan the list of mapped patient data for units of patient data assigned evidence types in the first set of evidence types; and populate a list of evidence with units of patient data extracted from the list of mapped patient data and linked to the first set of evidence types. The computer system can then selectively surface this list of patient evidence to the provider within the provider portal to support the first diagnosis provided by the provider.

### 12.1 Acceptance Score Prediction

In one variation, Blocks of the method S100 recite: in response to receiving selection of a subset of patient data, in the set of patient data, from the provider via the provider portal, appending the provider note with the subset of patient data linked to the diagnosis in Block S184; and calculating an acceptance score for the provider note based on the diagnosis and the subset of patient data, the acceptance score representing a likelihood of acceptance of the diagnosis for the patient during the encounter in Block S185.

In particular, in this variation, the computer system can calculate an acceptance score for a provider note—indicating a particular diagnosis for a particular encounter with a patient—based on the particular diagnosis and units of patient data selected by the provider for insertion into the provider note. For example, the computer system can calculate an acceptance score—representing a likelihood of acceptance of the provider note by an insurance company affiliated with the patient—for a provider note generated for an encounter with a patient. For example, the computer system can calculate an acceptance score represented by a percentage, a score between 1 and 10, a qualitative score (e.g., low, high, possible, probable), a binary score, etc. The computer system can compare the acceptance score to a threshold score—defined in the diagnostic model—to calculate acceptance and/or rejection of a particular provider note.

For example, the computer system can implement a single threshold score (e.g., a uniform threshold score) for each module in the population of modules. For example, the computer system can access a threshold score of 80%, defined for each module, in the population of modules, required to predict a diagnosis. Alternatively, the computer system can implement a variable threshold score defined for each individual module in the population of modules. For example, the computer system can: access a threshold score of 80%, defined for a first module and required to predict a first diagnosis; and access a threshold score of 50%, defined for a second module and required to predict a second diagnosis.

In this variation, the computer system can implement methods and techniques described above: to receive the diagnosis for a patient from a provider; to scan the list of mapped patient data for units of patient data supporting the diagnosis; and to transmit the notification to the provider (e.g., via the provider portal), the notification including the prompt to review each unit of patient data supporting the diagnosis (i.e., for insertion into the provider note). Then, in response to receiving selection of a subset of patient data—in the set of patient data—from the provider (e.g., via the provider portal), for insertion into the provider note, the computer system can: append the provider note with the subset of patient data linked to the diagnosis; and, based on the subset of patient data selected by the provider and the particular diagnosis, calculate an acceptance score for the provider note.

### 12.1.1 Acceptance Score Exceeding Threshold Score

In one variation, Block S187 of the method S100 recites, in response to the acceptance score exceeding a threshold score, verifying the provider note. Additionally or alternatively, in another variation, the computer system can prompt the provider to verify the provider note. For example, in this variation, in response to the acceptance score exceeding the threshold score, the computer system can: compile a set of provider note data including the diagnosis, the units of patient data supporting the diagnosis and selected by a provider, and a treatment pathway, in a set of treatment pathways, selected by the provider; and transmit a notification—including a prompt to verify the provider note for transmitting to an insurance company affiliated with the patient—to the provider (e.g., via the provider portal).

### 12.1.2 Acceptance Score Below Threshold Score

In one variation, Block S186 of the method S100 recites, in response to the acceptance score falling below a threshold score, withholding verification of the provider note. In particular, in this variation, in response to calculating an acceptance score falling below the threshold score, the computer system can withhold verification of a provider note for an encounter with a patient and suggest selecting additional patient data for insertion into the provider note. In particular, in this implementation, in response to receiving selection of a subset of patient data from the provider (e.g., via the provider portal) and calculating the acceptance score falling below the threshold score, the computer system can: withhold verification of the provider note for the encounter; and transmit a notification to the provider (e.g., via the provider portal) indicating the acceptance score falling below the threshold score and including a suggestion to select additional units of patient data from the set of patient data for insertion into the provider note. Then, in response to receiving selection of a second subset of patient data—in the set of patient data—from the provider (e.g., via the provider portal), for insertion into the provider note, the computer system can: append the provider note with the second subset of patient data linked to the diagnosis; and, based on the first subset of patient data (i.e., previously selected by the provider), the second subset of patient data selected by the provider, and the diagnosis, calculate second acceptance score for the provider note. In response to the acceptance score exceeding the threshold score the computer system can then verify the provider note.

### 12.2 Insufficient Evidence to Support Provider-Supplied Diagnosis

In one variation, Block S188 of the method S100 recites, in response to detecting absence of a unit of patient data corresponding to a target evidence type in the list of mapped patient data, identifying a medical test configured to yield patient data of the target evidence type. In particular, in this variation, the computer system can suggest retrieval of additional patient data to supplement a diagnosis supplied by the provider, such as in response to confidence in note acceptance falling below a threshold confidence and/or the list of mapped patient data missing a unit of patient data corresponding to a target evidence type required to support a diagnosis. For example, the computer system can: generate a notification indicating insufficient evidence included in a note for a particular diagnosis and including a suggestion and/or prompt to execute a particular test—defined by a module, in the diagnostic model, corresponding to the particular diagnosis—configured to retrieve additional health data that may support the particular diagnosis.

In this variation, the computer system can: receive a diagnosis from the provider (e.g., via the provider portal) for a patient, the diagnosis associated with the set of target evidence supporting the diagnosis including a first target evidence type and a second target evidence type; and extract a set of patient data—from the list of mapped patient data—including a first unit of patient data corresponding to the first target evidence type. The computer system can then generate and transmit a notification to the provider (e.g., via the provider portal) suggesting retrieval of additional health data for the patient in response to detecting absence of a second unit of patient data—in the list of mapped patient data—corresponding to the second target evidence type. More specifically, the computer system can: access a medical test defined for the second target evidence type, the medical test configured to yield a unit of patient data corresponding to the second target evidence type; and transmit the notification to the provider (e.g., via the provider portal) indicating insufficient evidence for the diagnosis and including a suggestion to execute the medical test with the patient.

For example, the computer system can receive an atrial fibrillation diagnosis from the provider (e.g., via the provider portal) for a patient during an encounter. The computer system can then scan the list of mapped patient data—associated with the patient—for a first unit of patient data corresponding to a first evidence type (e.g., a CHA2DS2-VASc score exceeding a threshold score and derived from patient health data including instances of prior stroke(s), patient diagnoses of diabetes, hypertension, heart failure, and/or vascular disease, patient age, patient sex, etc.) and second unit of patient data corresponding to a second evidence type (e.g., a target electrocardiogram (or “EKG”) reading depicting absence of P-waves, an irregular ventricular rate, or f waves discharging at a frequency of 350 to 600 beats/min) defined for the atrial fibrillation diagnosis.

The computer system can then: extract a first unit of patient data corresponding to a CHA2DS2-VASc score recorded for the patient and exceeding the threshold score; and detect absence of a second unit of patient data—corresponding to the target EKG reading. In response to detecting absence of the second unit of patient data, the computer system can: access a medical test defined for the target EKG reading within the atrial fibrillation module, the medical test configured to yield a patient indicator corresponding to the target EKG reading; and transmit a notification to the provider (e.g., via the provider portal) indicating insufficient evidence included in the provider note for the atrial fibrillation diagnosis and including a suggestion to execute the medical test with the patient.

Accordingly, the computer system can suggest retrieval of additional patient data that supports a provider-supplied diagnosis, in response to the list of mapped patient data missing a particular unit of patient data to aid the provider in supporting the diagnosis. Therefore, the computer system can reduce resources dedicated in generating provider notes and/or implementing post-hoc corrections due to unaccepted notes.

### 13. DIAGNOSIS PREDICTION

In one variation, as shown in FIG. 1C, Blocks of the method S100 recite: for a diagnosis, in a population of diagnoses defined in the diagnostic model, accessing a set of target evidence types supporting the first diagnosis in Block S190; and extracting a set of patient data, from the list of mapped of patient data, corresponding to the set of target evidence types in Block S181.

In one variation, the computer system can leverage the list of mapped patient data to predict a likelihood of a positive diagnosis for each diagnosis, in the population of diagnoses, defined in the diagnostic model. In particular, in this variation, the computer system can: access a first module, in the population of modules, associated with a first diagnosis and defining a first set of target evidence types; implement methods and techniques described above to populate a first list of evidence—including units of patient data extracted from the list of mapped patient data—linked to the first set of target evidence types defined for the first diagnosis; and, based on a correlation between the first set of target evidence types and the list of evidence, characterize a first confidence score for a positive diagnosis of the first diagnosis for the patient. Then, based on the first confidence score, the computer system can selectively present and/or withhold presentation of the first diagnosis to the provider within the provider portal. The computer system can then repeat this process for each diagnosis, in the population of diagnoses, to selectively present a set of missed diagnoses (or “suggested” diagnoses”) to the provider in (near) real-time during the encounter—such as within seconds of initiation of the encounter—for further review.

### 12.1 Confidence Score Derivation

In one variation, Block S195 of the method S100 recites deriving a confidence score for a positive diagnosis of the diagnosis for the patient based on a correlation between the set of target evidence and the set of patient data. In particular, in response to identifying units of patient data—represented in the list of mapped patient data and corresponding to target evidence types supporting a diagnosis—the computer system can derive a confidence score representing a confidence in the diagnosis for the patient based on the correlation between the patient data and the target evidence types.

For example, the computer system can derive the confidence score represented by a percentage, a score between 1 and 10, a qualitative score (e.g., low, high, possible, probable), a binary score, etc. The confidence score can be compared to a threshold score—defined in the diagnostic model—to calculate a confidence in a particular diagnosis for a patient. For example, the computer system can implement a single threshold score (e.g., a uniform threshold score) for each module in the population of modules. Alternatively, the computer system can implement a variable threshold score defined for each individual module in the population of modules.

### 13.2 Presenting the Diagnosis

In one variation, Blocks of the method S100 recite, in response to the confidence score exceeding the threshold score: appending a list of predicted diagnoses with the diagnosis in Block S194; populating a notification with the list of predicted diagnoses, the set of patient data linked to the diagnosis, and a prompt to review the list of predicted diagnoses in Block S182; and via the provider portal, transmitting the notification to the provider for review in Block S183. In response to flagging a diagnosis for further investigation by the provider, the computer system can selectively present the diagnosis to the provider via the provider portal. In particular, in response to flagging the diagnosis for further investigation, the computer system can: pair the diagnosis with units of patient data extracted from the list of mapped patient data and supporting the diagnosis; and present the diagnosis and the units of patient data within the provider portal, such as below a set of existing diagnoses previously entered and/or selected by the provider.

Accordingly, for each diagnosis in the population of diagnoses, the computer system can: scan the list of mapped patient data (e.g., all contents of the patient's health record, all recent and historical health data collected for this patient) for units of patient data supporting the diagnosis for the patient; and selectively flag and/or withhold flagging of the diagnosis as a predicted diagnosis based on identification of the units of patient data—in the list of mapped patient data—supporting the diagnosis. Therefore, the computer system can generate a robust list possible diagnoses for this patient and present this list of possible diagnoses—such as in a particular order based on urgency, confidence in diagnosis, relevancy to a current encounter, etc.—for review within the provider portal in (near) real-time.

### 14. CONCLUSION

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method comprising:
at a first time, during an encounter between a patient and a provider:
receiving a first patient indicator exhibited by the patient;
querying a diagnostic model for a first set of possible diagnoses, in a population of diagnoses, for the patient, each diagnoses in the first set of possible diagnoses associated with an evidence type analogous to the first patient indicator;
aggregating a first set of evidence types corresponding to the first set of possible diagnoses;
populating a first data request with a patient identifier of the patient, the first set of evidence types, and a first time period preceding the encounter;
transmitting the first data request to a health record database containing a corpus of historical patient data;
receiving a first set of patient data corresponding to the first set of evidence types responsive to the first data request; and
rendering the first set of patient data; and
at a second time, succeeding the first time, during the encounter:
populating a second data request, different from the first data request, with the patient identifier;
transmitting the second data request to the health record database;
receiving a second set of patient data responsive to the second data request; and
rendering the second set of patient data.

2. The method of claim 1, further comprising, at a third time, succeeding the second time, during the encounter:
populating a third data request with the patient identifier and a second time period preceding the first time period;
transmitting the third data request to the health record database;
receiving a third set of patient data responsive to the third data request; and
rendering the third set of patient data.

3. The method of claim 2:
wherein populating the first data request comprises populating the first data request with:
the patient identifier;
the first set of evidence types; and
the first time period comprising a one-year time period preceding the encounter;
wherein receiving the first set of patient data comprises receiving a first subset of patient data contained in a health record stored in the health record database and corresponding to the patient, the first subset of patient data:
corresponding to the first set of evidence types; and
recorded within the one-year time period preceding the encounter;
wherein rendering the first set of patient data comprises rendering the first set of patient data within a five-second time window following receipt of the first set of patient data;
wherein populating the second data request comprises populating the second data request with:
the patient identifier;
a second set of evidence types excluding the evidence type; and
the first time period comprising the one-year time period preceding the encounter;
wherein receiving the second set of patient data comprises receiving a second subset of patient data contained in the health record, the second subset of patient data recorded within the one-year time period preceding the encounter;
wherein rendering the second set of patient data comprises rendering the second set of patient data within a one-minute time window following receipt of the second set of patient data;
wherein populating the third data request comprises populating the third data request with:
the patient identifier; and
the second time period comprising an unbounded time period preceding the first time period;
wherein receiving the third set of patient data comprises receiving an entirety of the health record recorded within the unbounded time period preceding the first time period; and
wherein rendering the third set of patient data comprises rendering the third set of patient data within a two-minute time window following initiation of the encounter.

4. The method of claim 1:
further comprising:
for a first unit of patient data, in the first set of patient data, corresponding to a first evidence type:
detecting a first format of the first unit of patient data congruent with a first target format specified for the first evidence type; and
populating a list of mapped patient data with the first unit of patient data; and
for a second unit of patient data, in the first set of patient data, corresponding to a second evidence type:
detecting a second format of the second unit of patient data different from a second target format specified for the second evidence type;
extracting a set of metadata from the second unit of patient data;
accessing a set of mapping rules defined for patient data of the second evidence type; and
in response to the set of metadata corresponding to criteria defined by the set of mapping rules:
transforming the set of metadata into the second target format to generate a unit of mapped patient data; and
populating the list of mapped patient data with the unit of mapped patient data; and
wherein rendering the first set of patient data comprises rendering the list of mapped patient data.

5. The method of claim 1:
further comprising:
for a first unit of patient data, in the first set of patient data, corresponding to a first evidence type:
detecting a first format of the first unit of patient data congruent with a first target format specified for the first evidence type; and
populating a list of mapped patient data with the first unit of patient data; and
for a second unit of patient data, in the first set of patient data, corresponding to a second evidence type:
detecting a second format of the second unit of patient data different from a second target format specified for the second evidence type;
extracting a set of metadata from the second unit of patient data;
accessing a set of mapping rules defined for patient data of the second evidence type; and
in response to the set of metadata falling outside criteria defined by the set of mapping rules:
accessing a rule-generating model configured to autonomously generate mapping rules based on metadata extracted from units of patient data;
based on the rule-generating model and the set of metadata, calculating a confidence score for the second evidence type; and
in response to the confidence score exceeding a threshold score:
assigning the second evidence type to the second unit of patient data;
generating a second mapping rule linking the set of metadata to the second evidence type; and
appending the set of mapping rules with the second mapping rule; and
wherein rendering the first set of patient data comprises rendering the list of mapped patient data.

6. The method of claim 1:
further comprising:
accessing a provider specialty associated with the provider; and
querying the diagnostic model for a set of specialized diagnoses, in the population of diagnoses, associated with evaluation and treatment by the provider based on the provider specialty; and
wherein querying the diagnostic model for the first set of possible diagnoses comprises querying the diagnostic model for the first set of possible diagnoses contained within the set of specialized diagnoses.

7. The method of claim 6:
wherein identifying the provider specialty comprises identifying the provider specialty comprising endocrinology;
wherein querying the diagnostic model for the set of specialized diagnoses comprises querying the diagnostic model for the set of specialized diagnoses comprising a diabetes diagnosis, an osteoporosis diagnosis, and a hyperthyroidism diagnosis;
wherein receiving the first patient indicator comprises receiving the first patient indicator comprising fatigue exhibited by the patient during the encounter;
wherein querying the diagnostic model for the first set of possible diagnoses comprises querying the diagnostic model for the first set of possible diagnoses comprising the diabetes diagnosis and the hyperthyroidism diagnosis, each diagnosis in the first set of possible diagnoses associated with an evidence type analogous to fatigue; and
wherein aggregating the first set of evidence types comprises aggregating the first set of evidence types comprising:
a first subset of evidence types corresponding to the diabetes diagnosis; and
a second subset of evidence types corresponding to the hyperthyroidism diagnosis.

8. The method of claim 1:
further comprising, at the first time:
accessing a pre-encounter note generated prior to the encounter and associated with the patient;
extracting a second patient indicator from the pre-encounter note;
querying the diagnostic model for a second set of possible diagnoses in the population of diagnoses for the patient, each diagnosis, in the second set of possible diagnoses associated with a second evidence type analogous to the second patient indicator; and
aggregating a second set of evidence types corresponding to the second set of possible diagnoses;
wherein populating the first data request comprises further populating the first data request with the second set of evidence types; and
wherein receiving the first set of patient data comprises receiving the first set of patient data corresponding to the first set of evidence types and the second set of evidence types responsive to the first data request.

9. The method of claim 1:
further comprising, at the first time:
accessing an encounter type of the encounter; and
accessing a set of provider preferences specifying a second set of evidence types historically queried by the provider for encounters of the encounter type;
wherein populating the second data request comprises populating the second data request with the patient identifier, the first time period, and the second set of evidence types; and
wherein receiving the second set of patient data comprises receiving the second set of patient data corresponding to the second set of evidence types responsive to the second data request.

10. The method of claim 1, further comprising, at a third time succeeding the second time during the encounter:
receiving a first diagnosis for the patient from the provider via a provider portal executing on a computing device accessed by the provider, the first diagnosis associated with a set of target evidence types supporting the first diagnosis;
extracting a set of patient data, from the first set of patient data and the second set of patient data, corresponding to the set of target evidence types and supporting the first diagnosis for the patient;
generating a first notification comprising the set of patient data and a first prompt to review each unit of patient data, in the set of patient data, for insertion into a provider note generated for the encounter and indicating the first diagnosis for the patient;
transmitting the first notification to the provider via the provider portal; and
in response to receiving selection of a first subset of patient data, in the set of patient data, from the provider via the provider portal:
appending the provider note with the first subset of patient data linked to the first diagnosis;
calculating a first acceptance score for the provider note based on the first diagnosis and the first subset of patient data, the first acceptance score representing a likelihood of acceptance of the first diagnosis for the patient during the encounter; and
in response to the first acceptance score falling below a threshold score:
withholding verification of the provider note;
generating a second notification indicating the first acceptance score falling below the threshold score and comprising a second prompt to select additional patient data, in the set of patient data, for insertion into the provider note; and
transmitting the second notification to the provider via the provider portal.

11. The method of claim 10, further comprising, in response to receiving a second subset of patient data, in the set of patient data, insertion into the provider note from the provider:
appending the provider note with the second subset of patient data;
calculating a second acceptance score for the provider note based on the first diagnosis, the first subset of patient data, and the second subset of patient data; and
in response to the second acceptance score exceeding the threshold score, verifying the provider note.

12. The method of claim 10:
wherein receiving the first diagnosis for the patient comprises receiving the first diagnosis associated with the set of target evidence types supporting the first diagnosis comprising a first target evidence type and a second target evidence type;
wherein extracting the set of patient data comprises extracting the set of patient data comprising a first unit of patient data corresponding to the first target evidence type; and
further comprising, in response to detecting absence of a second unit of patient data corresponding to the second target evidence type in the first set of patient data and the second set of patient data:
identifying a medical test configured to yield patient data of the second target evidence type;
generating a third notification:
indicating insufficient evidence to support the first diagnosis; and
comprising a prompt to prescribe the medical test to the patient; and
transmitting the second notification to the provider via the provider portal.

13. The method of claim 1, further comprising, at the second time:
for a first diagnosis, in a population of diagnoses defined in the diagnostic model, accessing a set of target evidence types supporting the first diagnosis;
extracting a subset of patient data, from the first set of patient data and the second set of patient data, corresponding to the set of target evidence types;
calculating a confidence score for a positive diagnosis of the first diagnosis for the patient based on correspondence between the set of target evidence types and the subset of patient data; and
in response to the confidence score exceeding a threshold score:
appending a list of predicted diagnoses with the first diagnosis;
populating a first notification with the list of predicted diagnoses, the subset of patient data linked to the first diagnosis, and a first prompt to review the list of predicted diagnoses; and
transmitting the first notification to the provider for review.

14. A method comprising:
at a first time, preceding an encounter between a patient and a provider:
accessing a patient identifier of the patient from a patient list representing patients associated with the provider via a provider portal executing on a computing device accessed by the provider;
populating a first data request with the patient identifier, and a first time period preceding the encounter;
transmitting the first data request to a health record database containing a corpus of historical patient data;
receiving a first set of patient data responsive to the first data request; and
for each unit of patient data in the first set of patient data:
accessing a target format specified for the unit of patient data based on an evidence type corresponding to the unit of patient data; and
populating a list of mapped patient data with the unit of patient data in the target format; and
at a second time, succeeding the first time, during the encounter with the patient:
populating a second data request with:
the patient identifier; and
a second time period between the first time and the second time;
transmitting the second data request to the health record database;
receiving a second set of patient data responsive to the second data request;
for each unit of patient data in the second set of patient data:
accessing a target format specified for the unit of patient data based on an evidence type corresponding to the unit of patient data; and
populating the list of mapped patient data with the unit of patient data in the target format; and
rendering the list of mapped patient data within the provider portal.

15. The method of claim 14:
wherein populating the first data request comprises populating the first data request with:
the patient identifier; and
the first time period comprising a one-year time period preceding the encounter;
wherein rendering the first set of patient data comprises rendering the first set of patient data within a two-minute time window following receipt of the first set of patient data;
wherein populating the second data request comprises populating the second data request with:
the patient identifier; and
the second time period defined between the first time preceding the encounter and the second time during the encounter; and
wherein rendering the second set of patient data comprises rendering the second set of patient data within a five-second time window following receipt of the second set of patient data.

16. The method of claim 14:
further comprising:
for a first unit of patient data in the first set of patient data corresponding to a first evidence type:
detecting a first format of the first unit of patient data congruent with a first target format specified for the first evidence type; and
populating the list of mapped patient data with the first unit of patient data; and
for a second unit of patient data in the first set of patient data corresponding to a second evidence type:
detecting a second format of the second unit of patient data different from a second target format specified for the second evidence type;
extracting a set of metadata from the second unit of patient data;
accessing a set of mapping rules defined for patient data of the second evidence type; and
in response to the set of metadata corresponding to criteria defined by the set of mapping rules:
transforming the set of metadata into the second target format to generate a unit of mapped patient data; and
populating the list of mapped patient data with the unit of mapped patient data; and
wherein rendering the first set of patient data comprises rendering the list of mapped patient data within the provider portal.

17. The method of claim 14:
further comprising, at the first time:
accessing a pre-encounter note associated with the patient for the encounter and generated prior to the encounter;
extracting a patient indicator from the pre-encounter note;
querying a diagnostic model for a set of possible diagnoses in a population of diagnoses for the patient, each diagnosis in the set of possible diagnoses associated with an evidence type analogous to the patient indicator; and
aggregating a first set of evidence types corresponding to the set of possible diagnoses;
wherein populating the first data request comprises further populating the first data request with the first set of evidence types; and
wherein receiving the first set of patient data comprises receiving the first set of patient data corresponding to the first set of evidence types responsive to the first data request.

18. The method of claim 17:
further comprising, at the first time:
accessing a provider specialty associated with the provider; and
querying the diagnostic model for a set of specialized diagnoses, in the population of diagnoses, associated with evaluation and treatment by the provider based on the provider specialty; and
wherein querying the diagnostic model for the set of possible diagnoses comprises querying the diagnostic model for the set of possible diagnoses contained within the set of specialized diagnoses.

19. The method of claim 14:
further comprising:
for each unit of patient data in the first set of patient data, populating the list of mapped patient data with the unit of patient data in the target format in a first list; and
for each unit of patient data in the second set of patient data, populating the list of mapped patient data with the unit of patient data in the target format in a second list, presented above the first list; and
wherein rendering the list of mapped patient data comprises serving the list of mapped patient data to the provider via the provider portal at approximately the first time.

20. A method comprising:
at a first time preceding an encounter between a patient and a provider:
accessing a patient identifier of the patient via a provider portal executing on a computing device accessed by the provider;
populating a first data request with the patient identifier;
transmitting the first data request to a health record database containing a corpus of historical patient data;
receiving a first set of patient data responsive to the first data request; and
rendering the first set of patient data within the provider portal by, for each unit of patient data in the first set of patient data, populating a list of mapped patient data with a unit of mapped patient data corresponding to the unit of patient data; and
at a second time, succeeding the first time, during the encounter with the patient:
populating a second data request with:
the patient identifier; and
a time period defined between the first time and the second time;
transmitting the second data request to the health record database;
receiving a second set of patient data responsive to the second data request; and
rendering the second set of patient data for the provider to review by, for each unit of patient data in the second set of patient data, populating the list of mapped patient data with a unit of mapped patient data corresponding to the unit of patient data; and
at approximately the second time, serving the list of mapped patient data to the provider via the provider portal.
